(19) United States
(12) Patent Application Publication
(10) Pub. No.: US 2011/0218140 A1
(43) Pub. Date: Sep. 8, 2011

(54) BIODEGRADABLE THERAPEUTIC NANOPARTICLES CONTAINING AN ANTIMICROBIAL AGENT

(76) Inventors: Kenneth E. Gonsalves, Davidson, NC (US); Michael J. Bosse, Charlotte, NC (US); John Kent Ellington, Charlotte, NC (US); Michael C. Hudson, Charlotte, NC (US); James M. Horton, Charlotte, NC (US)

(21) Appl. No.: 12/866,939

(22) PCT Filed: Jan. 13, 2009
(86) PCT No.: PCT/US2009/030829

§ 371 Date: May 13, 2011

Related U.S. Application Data
(60) Provisional application No. 61/031,168, filed on Feb. 25, 2008.
(60) Provisional application No. 61/031,174, filed on Feb. 25, 2008.

(51) Int. Cl. A61K 38/14; A61K 31/43; A61P 31/04; A61P 19/00; B65D 85/00; B82Y 5/00

(52) U.S. Cl. 514/2.9; 514/2.3; 514/197; 206/459.5; 977/773; 977/906

(57) ABSTRACT

Compositions that include antimicrobial agents and biodegradable delivery-vehicles adapted to enter a cell and release the antimicrobial agents in the cell as they biodegrade. Also provided are compositions that include first and second delivery vehicles including first and second antimicrobial agents, wherein the first delivery vehicles are adapted to release the first antimicrobial agents at a rate that differs from that at which the second delivery vehicles release the second antimicrobial agents, articles of manufacture that include one or more biodegradable delivery vehicles, and methods of making and using the compositions to treat intracellular and/or extracellular infections are disclosed.

### CROSS REFERENCE TO RELATED APPLICATIONS

The presently disclosed subject matter claims the benefit of U.S. Provisional Patent Application Ser. Nos. 61/031,168 and 61/031,174, each filed Feb. 25, 2008; the disclosures of which are incorporated herein by reference in their entireties.

### TECHNICAL FIELD

The presently disclosed subject matter relates in part to compositions comprising antimicrobial-loaded delivery vehicles adapted to differentially release antimicrobial agents to target sites. The presently disclosed subject matter relates in part to compositions comprising a one or more delivery vehicles comprising one or more antimicrobial agents, wherein the delivery vehicles are adapted to release the antimicrobial agents intracellularly and/or extracellularly. Also provided are articles of manufacture comprising the compositions and methods for employing the compositions to treat microbial infections.

### BACKGROUND

Care of severe wounds is of primary concern to surgeons and first-responders in the United States of America and elsewhere. There is a long-felt and continuing need for research to improve the wound outcomes of patients who incur both high-energy soft tissue injuries and open fractures. Acute and chronic infections complicate recovery and limb reconstruction efforts. Clinical outcomes data from the Lower Extremity Assessment Project (LEAP) Study (Bosse et al., “An analysis of outcomes of reconstruction or amputation after leg-threatening injuries” New England Journal of Medicine, Vol. 347, pgs. 1924-1931 (2002)) show that these complications negatively impact the final functional outcome of patients with severe lower extremity trauma.

Several bacterial pathogens are likely to be encountered following severe injury, such that a broad-spectrum approach at antimicrobial treatment is desired. Several bacterial species that could enter soft tissue wounds and ultimately bone, have the ability to invade bone-forming cells (e.g., osteoblasts; Ellington et al., “Mechanisms of Staphylococcus aureus invasion of cultured osteoblasts”, Microb. Pathog., Vol. 26, pgs. 317-23 (1999); Hudson et al., “Internalization of Staphylococcus aureus by cultured osteoblasts”, Microb. Pathog., Vol. 19, pgs. 409-19 (1995); Reilly et al., “Internalization of Staphylococcus aureus by embryonic chicken osteoblasts in vivo”, Bone, Vol. 26, pgs. 63-70 (2000); Bosse et al., “Internalization of bacteria by osteoblasts in a patient with recurrent, long-term osteomyelitis: A Case Report,” J. Bone Joint Surg., pgs. 1343-1347 (2005); Ellington et al., “Intracellular Staphylococcus aureus and antibiotic resistance: implications for treatment of staphylococcal osteomyelitis,” J. Orthop. Res., Vol. 24, pgs. 87-93 (2006)) and dendritic cells (phagocytic leukocytes), and are therefore protected from most antibiotics while present in the intracellular environment of human cells.

What are needed, therefore, are new compositions, as well as methods for employing such compositions, to easily and efficiently treat microbial infections.

To address this need, the presently disclosed subject matter provides compositions comprising delivery vehicles that can be employed for localized treatment of infections.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, provided are compositions comprising an antimicrobial agent and a biodegradable delivery vehicle, wherein the biodegradable delivery vehicle is adapted to enter a cell and release the antimicrobial agent in the cell as it biodegrades. The biodegradable delivery vehicle can comprise a nanoparticle. The biodegradable delivery vehicle can comprise poly(lactic acid), poly(lactide-co-glycolide), polyanhydride, poly(1, 3-bis-(carboxyphenoxypropane):sebacic acid, or a combination thereof. The antimicrobial agent can be selected from the group including but not limited to vancomycin and nafcillin. The biodegradable delivery vehicle can comprise at least two different antimicrobial agents. The composition can be formulated for local administration to a site of infection, optionally topical administration. The composition can comprise a pharmaceutically acceptable carrier, diluent, or excipient.

In some embodiments, provided are article of manufactures comprising a biodegradable delivery vehicle comprising an antimicrobial agent, packaged in a hermetically sealed, sterile container, the container having a label affixed thereto, the label bearing printed material identifying the antimicrobial agent and providing information useful to an individual administering the biodegradable delivery vehicle to a subject in need thereof, wherein the biodegradable delivery vehicle is adapted to enter infected cells to thereby kill intracellular microbes present therein. The hermetically sealed, sterile container can comprise a pharmaceutically acceptable carrier, diluent, or excipient, and/or can further comprise one or more substances that when a sterile liquid is added to the hermetically sealed, sterile container reconstitutes a pharmaceutically acceptable carrier, diluent, or excipient.

In some embodiments, provided are methods for treating a microbial infection at a pre-determined site in a subject, or the possibility of a microbial infection. In some embodiments, the microbial infection is characterized by intracellular presence of a microbe in a subject. The methods can comprise administering to the subject at the pre-determined site a composition comprising an antimicrobial agent complexed to and/or within a biodegradable delivery vehicle, and further wherein the biodegradable delivery vehicle is adapted to enter a cell and release the antimicrobial agent in the cell as it biodegrades to thereby kill intracellular microbes if present in the cells. The microbial infection can comprise a bacterial infection. The bacterial infection can comprise a S. aureus infection. The cell can comprise a bone cell, optionally an osteoblast.

In some embodiments, provided are compositions comprising a first delivery vehicle comprising a first antimicrobial agent and a second delivery vehicle comprising a second antimicrobial agent, wherein the first delivery vehicle is adapted to release the first antimicrobial agent at a rate that differs from that at which the second delivery vehicle releases the second antimicrobial agent. The first delivery vehicle, the second delivery vehicle, or both can comprise a nanoparticle. The first delivery vehicle, the second delivery vehicle, or both can comprise poly(lactic acid), poly(lactide-co-glycolide), polyanhydride, poly(1, 3-bis-(carboxyphenoxypropane):sebacic acid, or a combination thereof. The first and the second delivery vehicles can comprise parts of a single structure. The at least one of the first antimicrobial agent and the second antimicrobial agent can be selected from a group including but not limited to vancomycin and nafcillin. The first antimicrobial agent and the second antimicrobial agent can be the same. The composition can be formulated for local administration to a site of infection, optionally topical administration. The composition can comprise a pharmaceutically acceptable carrier, diluent, or excipient.

In some embodiments, provided are article of manufactures comprising a first delivery vehicle comprising a first antimicrobial agent and a second delivery vehicle comprising a second antimicrobial agent, packaged in a hermetically sealed, sterile container, the container having a label affixed thereto, the label bearing printed material identifying the first antimicrobial agent and/or the second antimicrobial agent and providing information useful to an individual administering the first and second delivery vehicles to a subject in need thereof, wherein the first delivery vehicle is adapted to degrade rapidly in vivo in order to kill extracellular microbes, if any, present at a site on and/or in the subject, and the second delivery vehicle is adapted to enter infected cells present at the site, to thereby kill intracellular microbes. The hermetically sealed, sterile container can comprise a pharmaceutically acceptable carrier, diluent, or excipient, and/or can comprise one or more substances that when a sterile liquid is added to the hermetically sealed, sterile container reconstitutes a pharmaceutically acceptable carrier, diluent, or excipient.

In some embodiments, provided are methods for treating a pre-determined site in a subject for a microbial infection, or the possibility of a microbial infection. The microbial infection can be characterized by both extracellular and intracellular presence of a microbe in a subject. In some embodiments, the methods can comprise administering to the subject at the pre-determined site a composition comprising a first delivery vehicle comprising a first antimicrobial agent; and a second delivery vehicle comprising a second antimicrobial agent, wherein the first delivery vehicle is adapted to deliver the first antimicrobial agent in a burst in order to kill extracellular microbes, if present at the site, and the second delivery vehicle is adapted to enter cells present at the site, to thereby kill intracellular microbes if present in the cells. The microbial infection can comprise a bacterial infection, such as but not limited to a S. aureus infection. The site of infection can comprise a bone cell, optionally an osteoblast.

Accordingly, it is an object of the presently disclosed subject matter to provide improved antimicrobial compositions and related methods. This and other objects are achieved in whole or in part by the presently disclosed subject matter.

Other objects, features and advantages of the presently disclosed subject matter will become apparent from the following detailed description. It should be understood, however, that the detailed description and the specific examples, while indicating embodiments of the presently disclosed subject matter, are given by way of illustration only, since various changes and modifications within the spirit and scope of the presently disclosed subject matter will become apparent to those skilled in the art from this detailed description.

### BRIEF DESCRIPTION OF THE DRAWINGS

FIGS. 1 and 2 are confocal images of PLGA nanoparticles incorporated with Quantum Dots (QDs).

FIGS. 3 and 4 are confocal images of diffusion of QDs into osteoblasts.

FIGS. 5 and 6 are confocal images of intracellular diffusion of QD incorporated into mouse osteoblasts.

FIG. 7 is a scanning electron micrograph image of nanoparticles prepared by Scheme 1.

FIGS. 8A and 8B are scanning electron micrograph images of nanoparticles prepared according to Example 2.

FIG. 9A is a plot of cumulative drug release (%) versus time in hours, for release of nafcillin from nafcillin-PBS solution loaded in a dialysis bag with a molecular weight cut-off of 6 kDa at 37° C.

FIG. 9B is a plot of cumulative drug release (%) versus time in hours for release of nafcillin from nafcillin-PBS solution loaded in a dialysis bag with a molecular weight cut-off of 12-14 kDa at 37° C.

FIG. 10A is a plot of cumulative drug release (%) versus time in days for release of Nafcillin from PLGA 75:25 nanoparticles in PBS at 37° C.

FIG. 10B is a plot of cumulative drug release (%) versus time in days for release of Nafcillin from PLGA 50:50 nanoparticles in PBS at 37° C.

FIG. 11 is a bar graph showing viability of intracellular S. aureus in osteoblasts treated with nanoparticles or nanoparticles loaded with nafcillin.

FIG. 12 is a graph of anticipated release profiles for various embodiments of the presently disclosed subject matter. A: a “spike” in delivery; B: slower sustained release; and C: a “spike” plus sustained release. Each spray ensemble is calculated based on release profiles.

### DETAILED DESCRIPTION

The present subject matter will be now be described more fully hereinafter with reference to the accompanying Examples, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Unless otherwise defined, all technical and scientific terms used herein have the same meaning as commonly understood by one of ordinary skill in the art to which the presently disclosed subject matter belongs.

Following long-standing patent law convention, the terms “a”, “an”, and “the” refer to “one or more” when used in this application, including the claims, unless the context clearly indicates otherwise. Thus, for example, a reference to “a cell” can include multiple cells.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

Throughout this application, the term “about” is used to indicate that a value includes the inherent variation of error for the device, the method being employed to determine the value, or the variation that exists among the study subjects.

Throughout the specification and claims, a given chemical formula or name shall encompass all optical and stereoisomers as well as racemic mixtures where such isomers and mixtures exist.

### I. COMPOSITIONS

The presently disclosed subject matter provides in some embodiments nanoparticles loaded with one or more antimicrobial agent(s). Two types of nanoparticle-antimicrobial agent conjugates can be present in a representative composition of the presently disclosed subject matter, a fast-release particle (targeting extracellular microbes present at a pre-determined site, such as a wound), and a slow-release particle (targeting intracellular microbes that have already entered cells at the pre-determined site and remaining extracellular bacteria). Data demonstrating that this approach using antibiotic-loaded nanoparticles to enter infected osteoblasts results in killing of 100% of intracellular bacteria is presented herein below in the Examples.

Thus, provided in accordance with some embodiments of the presently disclosed subject matter is a composition comprising a first delivery vehicle comprising a first antimicrobial agent and a second delivery vehicle comprising a second antimicrobial agent, wherein the first delivery vehicle is adapted to release the first antimicrobial agent at a rate that differs from that at which the second delivery vehicle releases the second antimicrobial agent. Optionally, the first delivery vehicle is adapted to degrade rapidly at a site of delivery in order to kill extracellular microbes, if any, present at the site on and/or in the subject, and the second delivery vehicle is adapted to degrade and to release drug slowly and to enter infected cells present at the site, to thereby kill intracellular microbes and remaining extracellular bacteria.

Delivery time frames can be provided according to a desired treatment approach, which can include consideration of the microbe to be treated. By way of example and not limitation, the first delivery vehicle can deliver substantially all of the provided antimicrobial agent within 24 hours after administration wherein the second delivery vehicle can deliver a certain much smaller amount within the first 24 hours, first 3 days, first week, and substantially all within the first 2, 3, 4, 5, 6, or 7 weeks, as desired. Thus, the duration of the antimicrobial delivery can be altered with the chemistry of the nanoparticle.

Also, the first and the second delivery vehicles can comprise parts of a single structure. The first antimicrobial agent and the second antimicrobial agent can be the same.

The first and second delivery vehicles can comprise nano-, submicron-, and/or micron-sized particles. In some embodiments, one or both of the first and second delivery vehicles are about 50 nm to about 1 μm in their largest dimensions. Thus, in some embodiments the first delivery vehicle, the second delivery vehicle, or both can comprise a nanoparticle. As used herein, the terms “nano”, “nanoscopic”, “nanometer-sized”, “nanostructured”, “nanoscale”, and grammatical derivatives thereof are used synonymously and interchangeably and mean nanoparticles and nanoparticle composites less than or equal to about 1,000 nanometers (nm) in diameter.

The term “nanoparticle” as used herein denotes a carrier structure which is biocompatible with and sufficiently resistant to chemical and/or physical destruction by the environment of use such that a sufficient amount of the nanoparticles remain substantially intact after deployment at a site of interest. If the drug can enter the cell in the form whereby it is adsorbed to the nanoparticles, the nanoparticles must also remain sufficiently intact to enter the cell. Biodegradation of the nanoparticle is permissible upon deployment at a site of interest and/or entry of a cell. Nanoparticles can be particles ranging in size from 1 to 1,000 nm. Nanoparticles can have any diameter less than or equal to 1,000 nm, including 5, 10, 15, 20, 25, 30, 50, 75, 100, 200, 250, 30, 400, 500, 600, 750, 800, and 900 nm. Drugs, active agents, antibiotics or other relevant materials can be incubated with the nanoparticles, and thereby be adsorbed or attached to the nanoparticles.

As used herein, the term “biodegradable” means any structure, including but not limited to a nanoparticle, which decomposes or otherwise disintegrates after prolonged exposure to physiological conditions. To be biodegradable, the structure should be substantially disintegrated within a few weeks after introduction into the body.

Biodegradable biocompatible polymers can be used in drug delivery systems (Soppimath et al., J. Controlled Release, Vol. 70, pgs. 1-20 (2001); Song et al., J. Controlled Release, Vol. 43, pgs. 197-212 (1997)). The biodegradability and biocompatibility of poly(lactic acid) (PLA), poly(lactide-co-glycolide) (PLGA), and polyanhydrides (PAH) have been demonstrated. Some of the advantages of these materials include administration in high concentrations of the drug locally with low systemic levels, which reduces systemic complications and allergic reactions (Calhoun et al., Clin. Orthopaed. Related Res., Vol. 341, pgs. 206-214 (1997)). Additionally, no follow-up surgical removal is required once the drug supply is depleted (Mandal at al., Pharmaceut. Res., Vol. 19, pgs. 1713-1719 (2002)). Biodegradation occurs by simple hydrolysis of the ester backbone in aqueous environments such as body fluids. The degradation products are then metabolized to carbon dioxide and water (de Faria et al., Macromol. Symp., Vol. 229, pgs. 228-233 (2005)). Several techniques have been developed to prepare nanoparticles loaded with a broad variety of drugs using PLGA and to some extent with PAH (Lamprecht et al., Intl. J. Pharmaceut., Vol. 184, pgs. 97-105 (1999); Astete et al., J. Biomater. Sci. Polymer Edn., Vol. 17, pgs. 247-289 (2006); Hans et al., Solid State Mater. Sci., Vol. 6, pgs. 319-327 (2002); Kumar et al., Biomaterials, 25, pgs. 1771-1777 (2004); Laurencin et al., Biomaterials, Vol. 22, pgs. 1271-1277 (2001); Gonsalves et al., Biomaterials, Vol. 19, pgs. 1501-1505 (1998); Kwon et al., Colloids Surf. A, Vol. 182, pgs. 123-130 (2001)).

In some embodiments, the first delivery vehicle, the second delivery vehicle, or both can comprises poly(lactic acid), poly(lactide-co-glycolide), polyanhydride, poly(1, 3-bis-(carboxyphenoxypropane):sebacic acid, or any combination thereof. Certain particular percentages of starting materials can be employed if desired, such as but not limited to 50:50 PLGA, 75:25 PLGA, and the like. Materials and percentages of materials can be varied according to a desired delivery profile. Materials can include other biodegradable polymers such as polycaprolactones, polyorthoesters, polyphosphazenes etc.

Any suitable antimicrobial agent can be employed, as would be apparent to one of ordinary skill in the art upon a review of the instant disclosure. Indeed, the selection of an antimicrobial agent can depend on the microbe being targeted, for example, antibiotics, antifungals, and antivirals.

By way of example and not limitation, broad-spectrum antibiotics can be employed, including but not limited to beta-lactams (including but not limited to penicillins), cephalosporins, macrolides, tetracyclines, lincosamides, and aminoglycosides. In some embodiments, at least one of the first antimicrobial agent and the second antimicrobial agent is selected from the group including but not limited to vancomycin and nafcillin. In some embodiments, nafcillin-loaded nanoparticles are employed to target intracellular S. aureus. In some embodiments, nanoparticles are loaded with antibiotics such as but not limited to vancomycin, daptomycin, tobramycin, piperacillin/tazobactam or other drugs commonly used for the treatment of osteomyelitis or other microbial infections, alone or in combination.

In some embodiments, the composition can comprise a pharmaceutically acceptable carrier, diluent, or excipient. As used herein, the term “pharmaceutically acceptable” and grammatical variations thereof, as it refers to compositions, carriers, diluents and reagents, means that the materials are capable of administration to or upon a vertebrate subject without the production of undesirable physiological effects such as nausea, dizziness, gastric upset, fever and the like. In some embodiments, the “pharmaceutically acceptable” refers to pharmaceutically acceptable for use in human beings.

Compositions in accordance with the presently disclosed subject matter generally comprise an amount of the desired delivery vehicle-antimicrobial (which can be determined on a case-by-case basis), admixed with an acceptable pharmaceutical diluent or excipient, such as a sterile aqueous solution, to give an appropriate final desired concentration in accordance with the dosage information set forth herein, and/or as would be apparent to one of ordinary skill in the art upon a review of the instant disclosure, with respect to the antibiotic. Such formulations will typically include buffers such as phosphate buffered saline (PBS), or additional additives such as pharmaceutical excipients, stabilizing agents such as BSA or HSA, or salts such as sodium chloride. Such components can be chosen with the preparation of composition for local, and particularly topical, administration in mind.

In some embodiments, the composition is formulated for local administration to a pre-determined site (e.g. a site of infection, a site of injury, a site of surgery), optionally topical administration. Indeed, the presently disclosed compositions can be used to immediately treat injured subjects, and to target extracellular and intracellular bacterial pathogens, through a single or multiple applications (e.g., topical applications). Topical application can obviate the need for systemic antibiotic therapy, thereby mitigating the risk of selection for resistant bacteria associated with systemic application. Compositions for topical use can be provided in any suitable form, such as but not limited to a spray, an ointment, a gauze, a spray, or other formulation/article of manufacture. The presently disclosed subject matter can be applied as an ointment for example to replace BACITRACIN® ointment for surface infections; and it can be used as a nasal spray for sinus infection, acute or chronic. Techniques for preparing topical formulations, and other formulations, are generally well known in the art as exemplified by Remington's Pharmaceutical Sciences, (1980) (Osol, ed.) 16th Ed., Mack Publishing Company, Easton, Pa., incorporated herein by reference. In some embodiments, the composition is desirably stable at room temperature.

Also provided in accordance with some embodiments of the presently disclosed subject matter is an article of manufacture comprising a first delivery vehicle comprising a first antimicrobial agent and a second delivery vehicle comprising a second antimicrobial agent, packaged in a hermetically sealed, sterile container. Such articles of manufacture can be employed in any suitable setting, such as but not limited to in a first responder setting and a surgical setting.

The container can have a label affixed thereto. The label can bear printed material identifying the first antimicrobial agent and/or the second antimicrobial agent and can provide information useful to an individual administering the first and second delivery vehicles to a subject in need thereof.

In some embodiments, the hermetically sealed, sterile container further comprises a pharmaceutically acceptable carrier, diluent, or excipient. In some embodiments, the hermetically sealed, sterile container further comprises one or more substances that when a sterile liquid is added to the hermetically sealed, sterile container reconstitutes a pharmaceutically acceptable carrier, diluent, or excipient.

### II. METHODS OF TREATMENT AND/OR PROPHYLAXIS

The presently disclosed subject matter provides methods for treating a pre-determined site in a subject for a microbial infection, or the possibility of a microbial infection. In some embodiments, the method comprises administering to the subject at the pre-determined site a composition of the presently disclosed subject matter. As described herein above a composition of the presently disclosed subject matter can comprise: a first delivery vehicle comprising a first antimicrobial agent; and a second delivery vehicle comprising a second antimicrobial agent, wherein the first delivery vehicle is adapted to release the first antimicrobial agent at a rate that differs from that at which the second delivery vehicle releases the second antimicrobial agent.

In some embodiments, the microbial infection is characterized by both extracellular and intracellular presence of a microbe in a subject. Optionally, the first delivery vehicle is adapted to degrade rapidly at a site of delivery in order to kill extracellular microbes, if any, present at the site on and/or in the subject, and the second delivery vehicle is adapted to degrade and to release drug slowly and to enter infected cells present at the site, to thereby kill intracellular microbes.

In some embodiments, the microbial infection comprises a bacterial infection. In some embodiments, the bacterial infection comprises a S. aureus infection (including MRSA and CA-MRSA), other gram-positive bacteria, gram-negative bacteria, and/or anaerobes. In some embodiments, the microbial infection comprises an ear infection and/or an abdominal infection, although it is understood that the methods and compositions of the presently disclosed subject matter are not limited to use in only these types of infections.

In some embodiments, the pre-determined site for treatment comprises a wound or other injury. Thus, the presently disclosed methods can provide for the immediate treatment of injured patients, such as at the site of an accident and/or on a battlefield. In some embodiments, the pre-determined site for treatment comprises a site of a surgical procedure. In some embodiments, the pre-determined site of treatment comprises an infection or comprises a location where an infection might occur. Thus, prophylaxis of infection can be provided. In some embodiments, the pre-determined site for treatment comprises a bone cell, optionally an osteoblast.

In some embodiments, the presently disclosed methods comprise a topical application, which can replace systemic antibiotic therapy and its associated risk of selection for resistant bacteria. In some embodiments, the topical antibiotics can be applied to a wound by first-responders and at subsequent debridement surgeries. Hydrophilic bonding of the particles extracellularly and intracellular penetration of the particle can partner well with negative pressure wound therapy. Topical direct injury site therapy can be employed throughout the patient's surgical reconstructions. At the time of definitive wound closure, the particles can be placed in the tissues to provide antibiotic coverage. Additionally, this approach can replace the need to systemic peri-operative prophylactic antibiotics. Placed in the surgical wound at the time of incision and re-dosed at the time of wound closure, a constant effective level of local antibiotics can protect the surgical tissue bed. The duration of the antibiotic delivery can be altered with the chemistry of the delivery vehicles, which can be nanoparticles as disclosed herein above

The presently disclosed methods utilize in some embodiments nanoparticles loaded with an effective amount of antibiotics in a targeted, topical approach to improve antibiotic effectiveness in preventing soft tissue infections and osteomyelitis. This directly impacts the health and long-term recovery of injured patients and translates into improved initial care of surgical patients and for the care of patients with established infections of bone or soft tissues. The presently disclosed methods thus provide advantages to current standards and can be used in the care of high-risk elective implant procedures for prophylactic local antibiotic delivery. In some embodiments of the presently disclosed subject matter, antibiotic-loaded nanoparticles are used to treat bacterial infections such as but not limited to Staphylococcus aureus-infected osteoblasts, and S. aureus soft tissue infections and osteomyelitis. S. aureus is a capable bone pathogen, with adhesins that facilitate its binding to bone matrix (Boden et al., “Cloning and characterization of a gene for a 19 kDa fibrinogen-binding protein from Staphylococcus aureus,” Mol. Microbiol., Vol. 12, pgs. 599-606 (1994); Cheung et al., “Cloning expression and nucleotide sequence of a Staphylococcus aureus gene (fbpA) encoding a fibrinogen-binding protein,” Infect. Immun., Vol. 63, pgs. 1914-20 (1995); Flock et al., “Cloning and expression of the gene for a fibronectin-binding protein from Staphylococcus aureus,” EMBO. J., Vol. 6 2351-2357 (1987); Jonsson et al., “Two different genes encode fibronectin binding proteins in S. aureus: The complete nucleotide sequence and characterization of second gene,” Eur. J. Biochem. Vol. 202, pgs. 1041-1048 (1991); McDevitt et al., “Molecular characterization of the clumping factor (fibrinogen receptor) of Staphylococcus aureus”, Mol. Microbiol., Vol. 11, pgs. 237-48 (1994); McGavin et al., “Identification of a Staphylococcus aureus extracellular matrix-binding protein with broad specificity,” Infect. Immun., Vol. 61, pgs. 2479-85 (1993); Park et al., “Molecular cloning and expression of the gene for elastin-binding protein (ebpS) in Staphylococcus aureus,” J. Biol. Chem., Vol. 271, pgs. 15803-9 (1996); Patti et al., “Molecular characterization and expression of a gene encoding a Staphylococcus aureus collagen adhesion”, J. Biol. Chem., Vol. 267, pgs. 4766-72 (1992)) and toxin secretion capable of stimulating bone resorption (Nair et al., “Surface-associated proteins from Staphylococcus aureus demonstrate potent bone resorbing activity,” J. Bone Miner. Res., Vol. 10, pgs. 726-34 (1995)) via increasing osteoclast activity (Arora et al., “Effect of Staphylococcus aureus extracellular proteinaceous fraction in an isolated osteoclastic resorption assay,” J. Bone Miner. Res. Vol. 16, pgs. 158-61 (1998)).

S. aureus not only colonizes bone matrix, but is also internalized by osteoblasts. This has been demonstrated in vitro (Ellington et al., “Mechanisms of Staphylococcus aureus invasion of cultured osteoblasts,” Microb. Pathog., Vol. 26, pgs. 317-23 (1999); Hudson et al., “Internalization of Staphylococcus aureus by cultured osteoblasts”, Microb. Pathog., Vol. 19, pgs. 409-19 (1995); Reilly et al., “Internalization of Staphylococcus aureus by embryonic chicken osteoblasts in vivo”, Bone, Vol. 26, pgs. 63-70 (2000)) and in vivo (Bosse et al., “Internalization of bacteria by osteoblasts in a patient with recurrent, long-term osteomyelitis: A Case Report,” J. Bone Joint Surg., Vol. 87A, pgs. 1343-1347 (2005)). S. aureus is therefore capable of causing life-threatening infections when in the extracellular or intracellular environment. The mechanisms of invasion of S. aureus have been delineated, as has the immune response resulting from its infection of osteoblasts (Alexander et al., “Staphylococcus aureus and Salmonella enterica Serovar Dublin Induce Tumor Necrosis Factor-Related Apoptosis-Inducing Ligand Expression by Normal Mouse and Human Osteoblasts,” Infect. Immun., Vol. 69, pgs. 1581-1586 (2001); Bost et al., “Induction of colony-stimulating factor expression following Staphylococcus or Salmonella interaction with mouse or human osteoblasts,” Infect. Immun., Vol. 68, pgs. 5075-83 (2000); Bost et al., “Monocyte Chemoattractant Protein-1 Expression by Osteoblasts following Infection with Staphylococcus aureus or Salmonella”, J. Interferon Cytokine Res., Vol. 21, pgs. 297-304 (2001); Bost et al., “Staphylococcus aureus infection of mouse or human osteoblasts induces high levels of interleukin-6 and interleukin-12 production”, J. Infec. Dis. Vol. 180, pgs. 1912-20 (1999)). The ability of S. aureus to invade and survive within osteoblasts plays a role in the observation that greater than 80% of all cases of chronic osteomyelitis are caused by S. aureus (Waldvogel et al., “Osteomyelitis: A review of clinical features, therapeutic considerations and unusual aspects,” N. Eng. J. Med., Vol. 282, pgs. 198-206 (1970)). Intracellular invasion provides protection from the humoral immune response and several classes of antibiotics. This would further account for the persistence of disease despite what has been considered adequate surgical and antibiotic management.

S. aureus is also targeted for treatment in some embodiments of the presently disclosed subject matter for another reason. Traditional standard care treatment for gram-positive skin and soft tissue infections includes penicillins and cephalosporins (Lewis, RT, “Soft tissue infections,” World J. Surg. Vol. 22, pgs. 146-151 (1998)).

However, the effectiveness of these traditional antibacterial agents is becoming increasingly limited as the prevalence of antimicrobial-resistant in gram-positive bacteria, especially including methicillin-resistant S. aureus (MRSA), has increased sharply in recent years. Methicillin resistance in S. aureus often confers resistance to other beta-lactams and additional antibiotics, including macrolides, tetracyclines, lincosamides, and aminoglycosides, making the selection of antibiotic treatment more challenging. The incidence of multidrug-resistance and cross-resistance among gram-positive bacteria and widespread cross-resistance to antibiotic treatment is increasing at an alarming rate (Ellington et al., “Involvement of mitogen-activated protein kinase pathways in Staphylococcus aureus invasion of normal osteoblasts,” Infect. Immun., Vol. 69, pgs. 5235-42 (2001); Ellington et al., “Mechanisms of Staphylococcus aureus invasion of cultured osteoblasts”, Microb. Pathog., Vol. 26, pgs. 317-23 (1999); Schrum et al., “Functional CD40 Expression Induced Following Bacterial Infection of Mouse and Human Osteoblasts,” Infect. Immun., Vol. 71, pgs. 1209-16 (2003); Waldvogel et al., “Osteomyelitis: A review of clinical features, therapeutic considerations and unusual aspects,” N. Eng. J. Med., Vol. 282, pgs. 198-206 (1970); Herold et al., “Community-acquired methicillin-resistant Staphylococcus aureus in children with no identified predisposing risk,” JAMA, Vol. 279, pgs. 593-598 (1998); Petti et al., “Staphylococcus aureus bacteremia and endocarditis”, Infect. Dis. Clin. North Am., Vol. 16, pgs. 413-35 (2002); Martin et al., “The epidemiology of sepsis in the United States from 1979 through 2000”, N. Engl. J. Med., Vol. 348, pgs. 1546-54 (2003); Laupland et al., “Population-based study of the epidemiology of and the risk factors for invasive Staphylococcus aureus infections,” J. Infect. Dis., Vol. 187, pgs. 1452-9 (2003)).

Regarding skin, soft tissue, and bone infections, the increasing prevalence of MRSA is of primary concern (Rubin et al., “The economic impact of Staphylococcus aureus infection in New York City hospitals,” Emerg. Infect. Dis., Vol. 5, pgs. 9-17 (1999). For example, the SENTRY Program reported a methicillin-resistance rate of 34.2% among S. aureus isolates in the United States between 1997 and 1999, with a methicillin-resistance rate of approximately 30% among skin and soft tissue infection isolates (Diekema et al., “Survey of infections due to Staphylococcus species: Frequency of occurrence and antimicrobial susceptibility of isolates collected in the United States, Canada, Latin America, Europe, and the Western Pacific region for the SENTRY Antimicrobial Surveillance Program, 1997-1999,” Clin. Infect. Dis., Vol. 32, Suppl. 2, S114-S132 (2001)).

Although more often nosocomial (hospital-acquired), community-acquired MRSA infections are increasing rapidly (Herold at al., “Community-acquired methicillin-resistant Staphylococcus aureus in children with no identified predisposing risk,” JAMA, Vol. 279, pgs. 593-598 (1998); Morin et al., “Population-based incidence and characteristics of community-onset Staphylococcus aureus infections with bacteremia in 4 metropolitan Connecticut areas, 1998,” J. Infect. Dis., Vol. 184, pgs. 1029-1034 (2001); Groom et al., “Community-acquired methicillin-resistant Staphylococcus aureus in a rural American Indian community”, JAMA, Vol. 286, pgs. 1201-1205 (2001); Bukharie et al., “Emergence of methicillin resistant Staphylococcus aureus as a community pathogen”, Diag. Microbiol. Infect. Dis., Vol. 40, pgs. 1-4 (2001); Naimi et al., “Epidemiology and clonality of community-acquired methicillin-resistant Staphylococcus aureus in Minnesota, 1996-1998”, Clin. Infect. Dis., Vol. 33, pgs. 990-996 (2001); Charlebois et al., “Population-based community prevalence of methicillin-resistant Staphylococcus aureus in the urban poor of San Francisco”, Clin. Infect. Dis., Vol. 34, pgs. 425-433 (2002)). CA-MRSA strain USA 300 is employed in the Examples disclosed herein below.

Bacterial resistance increases the probability of treatment failures in patients with complicated skin and soft tissue infections, particularly for patients who have polymicrobial infections, and whose conditions were poor at the start of the antibiotic treatment (severe trauma) (Falagas et al., “Risk factors leading to clinical failure in the treatment of intra-abdominal or skin/soft tissue infections,” Eur. J. Clin. Microbiol. Infect. Dis., Vol. 15, pgs. 913-921 (1996)). Furthermore, antibiotics appropriate for MRSA might not be used promptly; the delay often results in less than optimal clinical outcomes and increased medical resource use and costs (Ibrahim et al., “The influence of inadequate antimicrobial treatment of bloodstream infections on patient outcomes in the ICU setting,” Chest, Vol. 118, pgs. 146-155 (1996)).

The presently disclosed subject matter includes but is not limited to the treatment of active infections, soft tissue or bone; the treatment of chronic infections, soft tissue or bone; the prevention of infections/surgical prophylaxis—orthopedic and general surgery and when foreign body implants are placed; to treat open/acute injuries, both soft tissue and bone; to treat pneumonia through spray or other formulations (which can be desirable for ventilator dependent patients in the ICU); and the use in the field such as medics applying treatment to a patient at the scene of the accident or on the way to a hospital through a gauze, a spray, or other formulation/article of manufacture. The presently disclosed subject matter can be applied as an ointment for example to replace BACITRACIN® ointment for surface infections; and it can be used as a nasal spray for sinus infection, acute or chronic. Additionally, the presently disclosed subject matter can be used to treat dental patients to prevent and treat bacterial infection. Treatment embodiments can include the use of a gauze or a spray to apply the nanoparticles. The presently disclosed subject matter can be applied to surgical implants (such as but not limited to orthopaedic hardware/arthroplasty, abdominal mesh, defribillators, etc), bone grafts (osteoconductive and osteoinductive) and also in conjunction with current and novel boney reconstruction technology. Application of the compositions of the presently disclosed subject matter to implants can be done by the manufacturer and/or a surgeon or other medical personnel.

Warm-blooded vertebrates comprise representative subjects for treatment in accordance with the methods of the presently disclosed subject matter. Therefore, the presently disclosed subject matter concerns mammals and birds.

Provided is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, provided is the treatment of livestock, including, but not limited to, domesticated swine (pigs and hogs), ruminants, horses, poultry, and the like.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Preparation of PLA and PLGA Nanoparticles

Staphylococcus aureus (S. aureus) is the major causative agent of osteomyelitis (bone inflammation) accounting for almost 80% of all cases of human disease (Ellington at al., J. Bone Joint Surg. Br., Vol. 85, No. 6, pgs. 918-21 (2003)). These bacteria are characterized by their high affinity to bone, rapid induction of osteonecrosis (bone death) and resorption of bone matrix (Lucke at al., J. Biomed. Mater. Res. B Appl. Biomater., Vol. 67, No. 1, pgs. 593-602 (2003)). While S. aureus is generally considered to be an extracellular pathogen, S. aureus has the ability to adhere to and persist within osteoblasts (bone-forming cells) (Ellington et al., Microb. Pathog., Vol. 26, No. 6, pgs. 317-23 (1999)). S. aureus-mediated osteomyelitis is often chronic in nature and highly resistant to antibiotic therapy. Therefore, it is possible that the ability of bacteria to persist intracellularly offers the bacteria a route to evade antibiotic treatment, which could subsequently lead to recurrent infections. Nanoparticles tagged to drugs could better facilitate movement of antibiotics such as nafcillin, vancomycin and daptomycin across the osteoblast cell membrane and ensure better dug delivery to the intracellular bacteria. Thus, among other aspects, the present Example examined the ability of quantum dots and quantum dots tagged to nanoparticles to enter live bone-forming cells.

Antibiotic-encapsulated PLA and PLGA nanoparticles were prepared by the single emulsion-solvent evaporation technique. Different PLA and PLGA systems were prepared, varying the copolymer composition and the amount of the surfactant polyvinyl alcohol. Characterization and drug loading studies were performed by UV-Visible spectrophotometry, dynamic light scattering, and scanning electron microscopy (SEM).

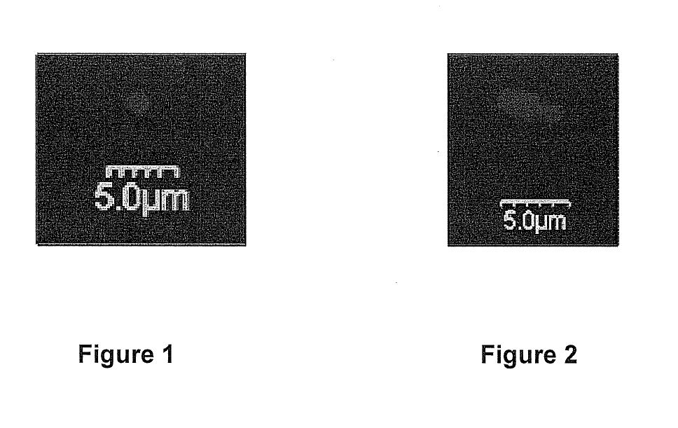

Simultaneously, in order to model the diffusion of the nanoparticles within the osteoblast cells, quantum dots (QDs), such as functionalized InGaP/ZnS and polymer encapsulated InGaP/ZnS nanoparticles, were added to confluent cultures of primary mouse osteoblasts. Following PREFER™ fixation, cultures were examined via confocal microscopy. QDs were clearly visible within osteoblasts. See FIGS. 1-6.

PLGA nanoparticles using several copolymer molecular weights were prepared by the single emulsion-solvent evaporation technique. Polyvinyl alcohol (PVA) was used because nanoparticles using this emulsifier are relatively uniform and smaller in size, and are easy to redisperse in aqueous medium (Panyam et al., Advanced Drug Delivery Reviews, Vol. 55, pgs. 329-347 (2003)). PLGA nanoparticles were loaded with nafcillin.

Quantum dots (QDs) are a new class of flourophores excitable over abroad wavelength range stretching from the UV and up to slightly less than their emission peak. They have narrow, size-tunable, emission bands, and are resistant to photobleaching (Lagerholm et al., Nanoletters, Vol. 4, pgs. 2019-2022 (2004)). Their biological applications in a variety of in vitro and in vivo procedures (Bruchez et al., Science, Vol. 281, pgs. 2013-2016 (1998); Larson et al., Science, Vol. 300, pgs. 1434-1436 (2003)) including methods of labeling cells with them have been reported (Jaiswal et al., Nature Biotechnology, Vol. 21, pgs. 47-51(2003); Rosenthal et al., J. Am. Chem. Soc., Vol. 124, pgs. 4586-4594 (2002)).

In/Ga/P amine functionalized QDs were used in order to model the diffusion of the nanoparticles within the osteoblast cells. The QD included an In/Ga/P core surrounded by a ZnS shell and a PEG lipid coating to make it water soluble. The lipid coat is functionalized with an amine linker group for subsequent conjugation to other bio-molecules, such as antibiotics. In addition, PLGA encapsulated InGaP/ZnS nanoparticles were added to confluent cultures of primary mouse osteoblasts. Following Prefer fixation, cultures were examined via confocal microscopy. QDs were clearly visible within infected osteoblasts.

Nanoparticles of poly(lactide-co-glycolide) were prepared by single emulsion solvent evaporation technique. The lactide content of the copolymers varied from 50, 75 to 100%. The effect of the stabilizer in the nanoparticle size was studied by using different amounts of polyvinyl alcohol (0, 0.5,1, 2, and 2.5%). Also nanoparticles loaded with nafcillin were prepared using the same technique. Finally, nanoparticles loaded with amine functionalized quantum dots composed of an In/Ga/P core surrounded by a ZnS shell and a PEG lipid coating functionalized with an amine linker group were prepared.

Materials: poly(dl-lactic acid) (PLA) MW 6,00-16,000, poly(dl-lactic-coglycolic acid; PLGA) at different ratios: 50:50 MW 12,000-16,000 and 75:25 MW˜20,000, and polyvinyl alcohol (PVA) were obtained from Polysciences, Warrington, Pa., United States of America. Dichloromethane (DCM), biotech grade, dimethyl sulfoxide (DMSO), spectrophotometric grade, and acetone, HPLC grade were obtained from Sigma-Aldrich, St. Louis, Mo., United States of America. Double deionized water, molecular biology grade (DDIW) was obtained from VWR. Nafcillin was obtained from Sandoz, Princeton, N.J., United States of America. Quantum dots (QD), T2-MP InGaP/ZnS Amine Macoun Red, 650 nm was obtained from Evident Technologies, Troy, N.Y., United States of America.

Nanoparticles preparation: PLGA was dissolved in 10 mL of the organic phase, DCM/acetone (8:2, v/v) at a concentration of 3.0%. For nanoparticles loaded with nafcillin, the drug was dissolved in 10 mL of the co-solvent acetone at its maximum solubility, a few drops of DDIW was added to achieve complete dissolution, the concentration was 1% w/v, both organic solutions were mixed together using a vortex mixer for 3 minutes. For nanoparticles loaded with QD, 3 mL of QD solution in DDIW at a concentration of 9×10⁻⁴ nmol/mL was prepared, and then added to the PLGA organic phase, and mixed using a vortex mixer for 3 minutes.

Twenty (20) mL of aqueous PVA solution was prepared at concentrations of 0.5, 1, 2, and 2.5% w/v. The oil-water (O/W) emulsion was obtained by pouring the organic phase into the aqueous phase, mixing with a vortex mixer for 1 minute and then sonicating with a probe at 90 W for 15 minutes over an ice bath. The organic solvents were allowed to evaporate while being stirred at atmospheric pressure. The solidified nanoparticles were collected by ultracentrifugation, first at 10 RPM for 15 minutes to eliminate the big nanoparticles, and then washed three times with DDIW at 35,000 RPM for 30 minutes to remove PVA and free drug. The final product was dried by lyophilization.

Particle size measurements: 0.1 mg of nanoparticles were suspended in 5 mL of DDIW, suspension was sonicated for 5 minutes, and the particle size was measured by Dynamic Light Scattering (DLS), using a Laser Light Scattering, 90Plus/BI.MAS Multi Angle Particle Sizing, Brookhaven Instruments Corporation, Holtsville, N.Y., United States of America.

Surface morphology of the nanoparticles was analyzed by Scanning Electron Microscopy (SEM) on a JEOL JSM 6460 LV Scanning Electron Microscope, available from JEOL USA Inc., Peabody, Mass., United States of America.

Drug loading measurements: Amount of nafcillin in PLGA nanoparticles was determined by UV-Visible Spectrophotometry. A calibration curve was prepared using standard solutions of nafcillin in DMSO at different known concentrations. Nafcillin loaded nanoparticles samples were dissolved in DMSO and the absorbance was measured at 324 and 335 nm. Absorbance values were correlated with drug concentrations using the calibration curve.

Osteoblasts experiments: Primary mouse osteoblasts were isolated from mouse neonatal calvariae by sequential protease and collagenase digestions as described (Alexander et al., BMC Microbiol., Vol. 3, pg. 5 (2003)). Cells were seeded on a cover slip in 24-well cluster plates and incubated at 37° C. in a 5% CO₂ incubator. Once the cells reached confluency, cells were washed once with Hank's balanced salt solution (HBSS) and were treated with quantum dots alone or quantum dots tagged to nanoparticles suspended in 1× Phosphate-buffered saline (PBS). Following different time periods of incubation (1, 2 hours), the quantum dots with and without nanoparticles were removed and rinsed twice with 1X PBS. Cell layers were rinsed twice with 1X PBS and were fixed using Prefer fixative or DMSO for 20 minutes. The fixative was then removed and the cells were washed twice with 1×PBS. Cells were counterstained with 4′-6-Diamidino-2-phenylindole (DAPI) for 10 minutes so as to visualize the nucleus, with stains blue with DAPI. The DAPI stain was then removed and cells were washed twice with 1×PBS. Finally, the extent of fluorescence present in the cells was visualized using a FLUOVIEW FV500 confocal laser scanning biological microscope (Olympus America Inc., Melville, N.Y., United States of America).

Synthesis and characterization of nanoparticles incorporating QDs: Nanoparticles containing quantum dots were synthesized by a single-emulsion solvent evaporation technique depicted in Scheme 1.

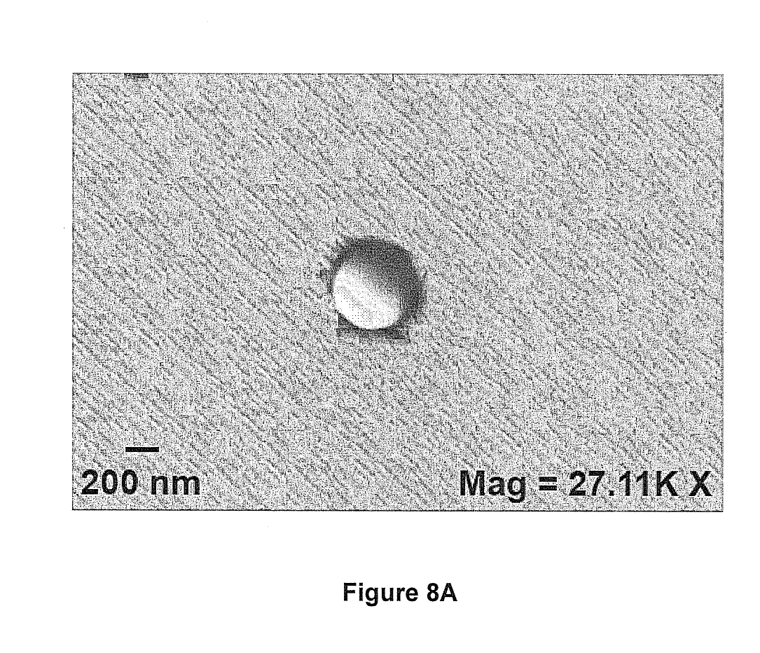

A summary of the preparation and particle size characterization of nanoparticles of PLGA loaded with QD T2-MP InGaP/ZnS amine functionalized are presented in Table 1 below. FIG. 7 is a scanning electron micrograph (SEM) image of nanoparticles produced through the strategy depicted in Scheme 1 below.

TABLE 1
PLGA-QD NANOPARTICLES PREPARATION AND
CHARACTERIZATION

| PLGA | PLGA | PVA | Particle size/ | QD Loading |
|---|---|---|---|---|
| Composition | (mg) | (mg, %) | PD (nm) | (%) |
| 50:50 | 313 | 116, 0.5 | 263 (0.13) | 9 × 10⁻⁴ nmol/mL |
| 75:25 | 304 | 107, 0.5 | 301 (0.20) | 9 × 10⁻⁴ nmol/mL |
| 100:0 | 305 | 107, 0.5 | 228 (0.18) | 9 × 10⁻⁴ nmol/mL |

As mentioned above nanoparticles tagged to drugs could better facilitate movement of antibiotics such as nafcillin, vancomycin and daptomycin across the osteoblast cell membrane and ensure better drug delivery to the intracellular bacteria. This Example examined the ability of quantum dots and quantum dots incorporated in nanoparticles to enter live bone-forming cells.

Primary mouse osteoblast cells were treated with quantum dots alone or quantum dots tagged to PLGA nanoparticles. After different incubation periods (1, 2 hours) and Prefer fixation the cultures were examined via confocal microscopy to observe the extent of fluorescence present in the cells.

The protocol followed for confocal microscopy is explained as follows:

1. Seed cells on cover slips in 24 well dishes.

2 Once the cells reach 80% confluence, remove media and wash 1×HBSS, Add 500 μL of OBGM (Osteoblast growth medium) to each well.

3. Suspensions of nanoparticles loaded with quantum dots (PLGA-QD) in 10 mL of OBGM that were resonicated for half an hour. (3 μL of stock QD* was added to PLGA to make PLGA tagged nanoparticles)

4. After 2 hours, remove PLGA-QD solutions and wash twice with 1×PBS (Phosphate buffered saline solution)

5. Add 500 μL of fixative solution (PREFER™ brand) and incubate for 20 minutes at room temperature. Cover the plates in aluminum foil.

6. After 20 minutes, remove fixative solution and wash twice with 1×PBS.

7. Add DAPI (4′,6-diamidino 2-phenylindole dihydrochloride) stain (40 μL/mL) for 5 minutes at room temp and shake gently. Wash twice with 1×PBS.

8. Remove the cover slip (use a 25 gauge needle as a lever and lift the cover slip off the bottom of the well using a sharp forceps).

9. Invert the cover slip (such that cells are facing down) on to one drop of aqueous mounting media placed on a microscopy slide.

10. Let the slide dry flat briefly and store flat at 4° C. overnight.

11. Coat the cover slip with nail polish the next day so as to seal the area around the slip.

### * QD: InGaP/ZnS Amine EviTag, Macoun Red, 650 nm

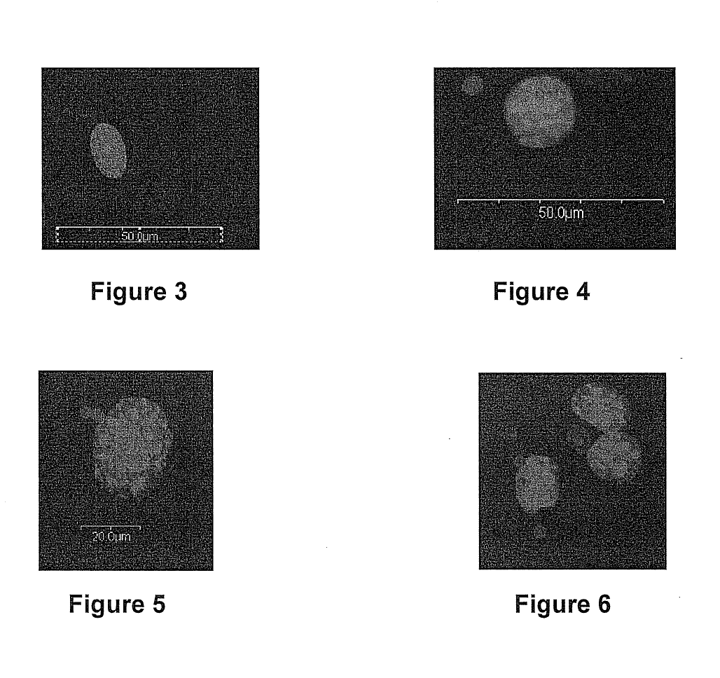

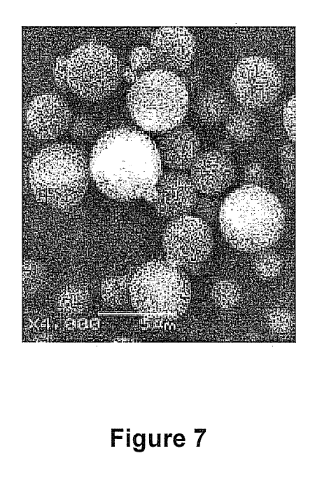

Referring again to the Figures, FIGS. 1 and 2 are confocal images of PLGA nanoparticles incorporated with QD, and in FIG. 1, PLGA 100:0, and in FIG. 2, PLGA 75:25, aqueous suspension of polylactide nanoparticles. FIGS. 3 and 4 show the intracellular diffusion of QDs into osteoblasts. FIG. 3 shows an intact osteoblast and FIG. 4 shows a lysed osteoblast with a round nucleus. Pin size spots correspond to QDs inside the nucleus. QDs are also evident outside the lysed cell. FIGS. 5 and 6 are confocal images of intracellular diffusion of QD incorporated into the mouse osteoblast. FIG. 5 shows osteoblast-PLGA 50:50 nanoparticle loaded with QD. FIG. 6 shows osteoblast-PLGA 75:25 nanoparticle loaded with QD.

Based on the QD data above, nanoparticles of poly(lactide-co-glycolide) were prepared by single-emulsion solvent evaporation technique. Three different copolymer compositions were used, and the amount of the emulsifier, PVA, was changed at different degrees. Nanoparticles were loaded with nafcillin. The particle size of the nanoparticles dispersions and drug loading of the dry nanoparticles were determined, and results are shown in Table 2.

TABLE 2
PLGA NANOPARTICLES LOADED WITH NAFCILLIN
PREPARATION AND CHARACTERIZATION

| PLGA | PLGA | Nafcillin | PVA | Particle size/ | Drug Loading |
|---|---|---|---|---|---|
| Composition | (mg) | (mg) | (mg, %) | PD | (%) |
| 50:50 | 482 | 0 | 162, 0.5 | 334 (0.24) | 0 |
| 50:50 | 407 | 139 | 141, 0.5 | 155 (0.20) | 43 |
| 50:50 | 305 | 100 | 209, 1.0 | 179 (0.12) | 34 |
| 50:50 | 308 | 107 | 404, 2.0 | 126 (0.12) | 29 |
| 50:50 | 312 | 105 | 514, 2.5 | 106 (0.12) | 23 |
| 75:25 | 314 | 0 | 109, 0.5 | 252 (0.10) | 0 |
| 75:25 | 304 | 107 | 103, 0.5 | 175 (0.11) | 45 |
| 75:25 | 308 | 106 | 214, 1.0 | 150 (0.18) | 29 |
| 75:25 | 302 | 107 | 402, 2.0 | 234 (0.21) | 33 |
| 75:25 | 306 | 105 | 507, 2.5 | 111 (0.14) | 16 |
| 100:0 | 308 | 0 | 110, 0.5 | 300 (0.03) | 0 |
| 100:0 | 302 | 107 | 103, 0.5 | 178 (0.19) | 11 |
| 100:0 | 316 | 110 | 201, 1.0 | 176 (0.22) | 4 |
| 100:0 | 312 | 111 | 401, 2.0 | 125 (0.23) | 6 |
| 100:0 | 310 | 111 | 516, 2.5 | 100 (0.15) | 3 |

In all three copolymer compositions, optimum combination of particle size and nafcillin loading was obtained when 0.5% of PVA was used to prepare the PLGA nanoparticles. The drug-loaded nanoparticle size when 0.5% PVA was used was slightly smaller than that of unloaded PLGA nanoparticles. The hydrophobic character of nafcillin, due to the aromatic rings in the molecular structure, might decrease the interfacial tension between the organic and aqueous phase, which results in an increase of the area to volume ratio and thus in smaller particles (Varela et al., Journal of Chemical Physics, Vol. 114, No. 17, pgs. 7682-7687 (2001); Zweers et al., Journal of Controlled Release, Vol. 114, No. 3, pgs. 317-324 (2006)).

The PLGA nanoparticles prepared in this Example were spherical in shape, with particle size in the range of 200 nm. The best results in terms of particle size and drug loading was obtained when 0.5% of the stabilizer PVA was used. Fluorescent confocal microscopy verified that the QDs diffused inside of the osteoblast and were largely partitioned between the nucleus and cytoplasm. Finally, PLGA nanoparticles loaded with QDs were located inside the osteoblast cell and in the periphery of the nucleus.

### Example 2

### Production of Nafcillin-loaded Nanoparticles

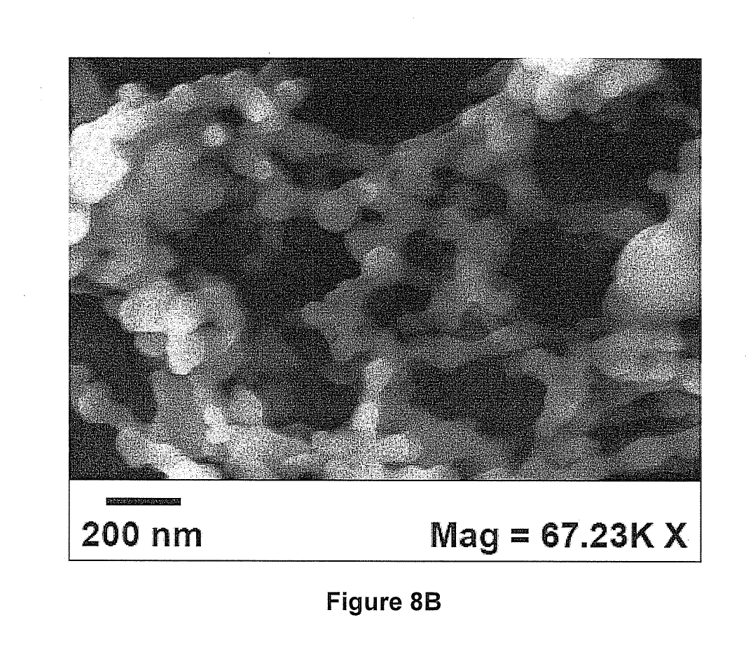

Nafcillin-loaded PLGA nanoparticles were prepared by a single emulsion method and their properties are given in Table 3. SEM images are provided in FIGS. 8A and 8B. FIGS. 8A and 8B show that nanoparticles formed by the solvent evaporation method are spherical in shape and with a smooth surface.

TABLE 3
PROPERTIES OF NAFCILLIN-LOADED NANOPARTICLES

| | Type | Weight | Weight | Weight | | | Drug |
|---|---|---|---|---|---|---|---|
| Sam- | of | of | of | of | | | Loading |
| ple | Poly- | PLGA | PVA | Nafcillin | Particle | Zeta | (%, |
| No. | mer | (mg) | (mg) | (mg) | (nm) | (mV) | w/w) |
| G1 | PLGA | 900 | 305 | 0 | 373 | −45 | n.a. |
| | 50:50 | | | | | | |
| G1.1 | PLGA | 910 | 306 | 310 | 140 | −78 | 10 |
| | 50:50 | | | | | | |
| G1.2 | PLGA | 908 | 305 | 300 | 168 | −77 | 09 |
| | 50:50 | | | | | | |
| G1.3 | PLGA | 300 | 100 | 100 | 160 | −66 | 10 |
| | 50:50 | | | | | | |
| G2 | PLGA | 905 | 102 | 0 | 280 | −45 | n.a. |
| | 75:25 | | | | | | |
| G2.1 | PLGA | 905 | 300 | 305 | 162 | −84 | 07 |
| | 75:25 | | | | | | |
| G2.2 | PLGA | 903 | 300 | 305 | 207 | −82 | 11 |
| | 75:25 | | | | | | |
| G2.3 | PLGA | 302 | 101 | 100 | 180 | −73 | 12 |
| | 75:25 | | | | | | |

A dialysis method was used in the measurement of in vitro drug release. Drug release studies were done under sink conditions; the volume of the buffer was kept large (outer sink) compared to the volume of drug-loaded nanoparticles suspension in the dialysis bag. The (theoretical) situation of infinite dilution is known as a perfect sink; however, even though perfect sink conditions are never attainable in practice, they are the only situation in which a true release profile can be measured. For a given matrix volume, an increase in liquid volume or volume ratio will promote drug release due to the lower drug concentrations in the medium. The system developed was continuously agitated, resulting in the faster diffusion of drug through the dialysis membrane to the outer sink.

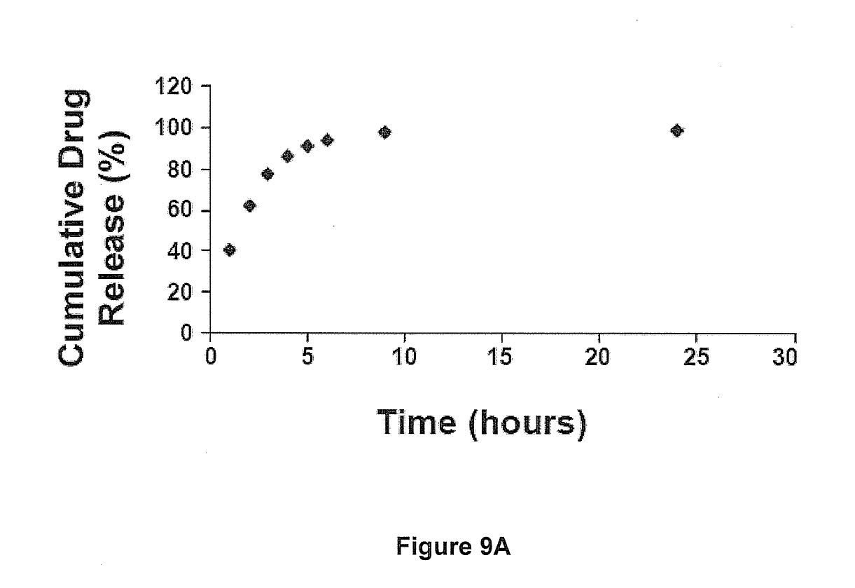

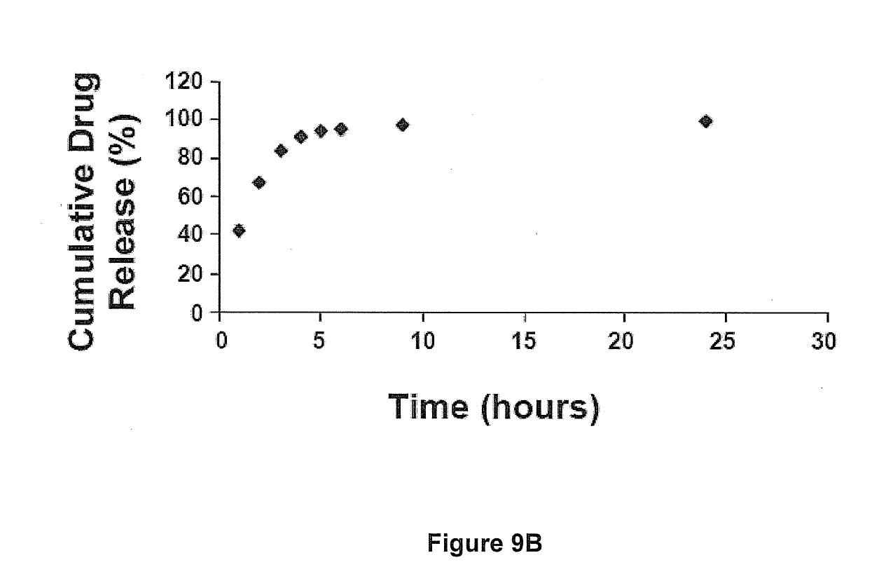

The release of nafcillin from nafcillin-PBS solution loaded in a dialysis bag is shown in FIGS. 9A and 9B. FIG. 9A is a plot of cumulative drug release (%) versus time in hours, for release of nafcillin from nafcillin-PBS solution loaded in a dialysis bag with a molecular weight cut-off of 6 kDa at 37° C. FIG. 9B is a plot of cumulative drug release (%) versus time in hours for release of nafcillin from nafcillin-PBS solution loaded in a dialysis bag with a molecular weight cut-off of 12-14 kDa at 37° C. The release of nafcillin from nafcillin-PBS solution without polymer nanoparticles was rapid, since 97% of the drug was released within the first nine (9) hours. This shows that the transport of drug through the dialysis membrane is not a rate-limiting factor.

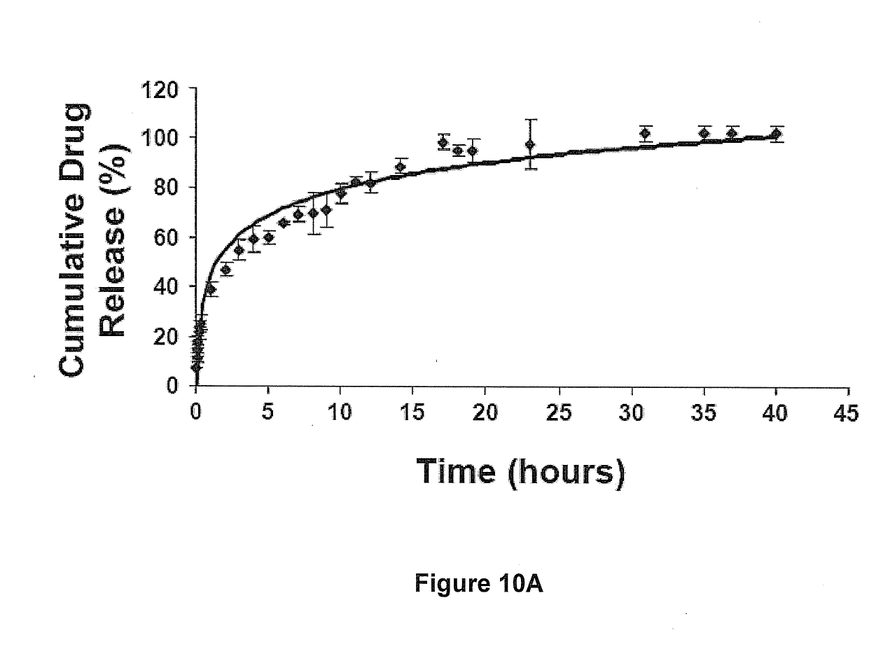

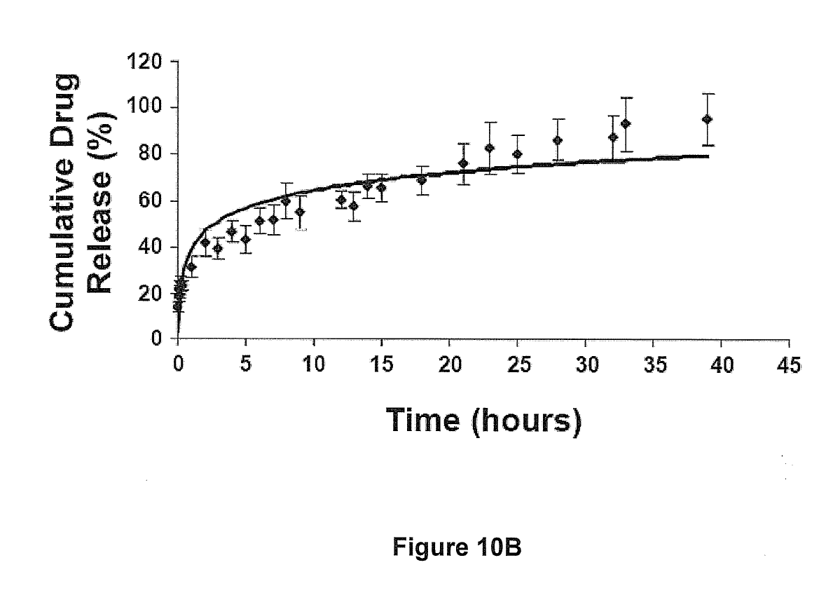

FIGS. 10A and 10B show the release of nafcillin from PLGA 75:25 and PLGA 50:50 loaded nanoparticles, respectively. FIG. 10A is a plot of cumulative drug release (%) versus time in days for release of Nafcillin from PLGA 75:25 nanoparticles in PBS at 37° C. FIG. 10B is a plot of cumulative drug release (%) versus time in days for release of nafcillin from PLGA 50:50 nanoparticles in PBS at 37° C. A biphasic release profile is evident. In the first phase of release (burst release) 42-47%of the drug was released within 48 hours of study. The entire drug was released within 35-40 days. The initial burst release was probably due to the drug that was present close to the surface of the nanoparticle. It was therefore released through a short diffusion pathway.

Biodegradable polymers can be arbitrarily classified into two groups—bulk eroding (homogeneous) and surface eroding (heterogeneous) polymers. In the case of surface eroding polymers, degradation is much faster than water intrusion into the polymer bulk. Therefore, erosion affects only the surface and not the inner parts of the matrix. In bulk eroding polymers water uptake by the system is much faster than polymer degradation. This results in the hydration of entire polymer and cleavage of polymer chains throughout. Therefore, the erosion process is not confined to the outer surface. PLGA is a bulk eroding polymer. The second phase of slow release was due to simultaneous polymer degradation and drug diffusion. This release profile is an aspect of the presently disclosed subject matter, since an initial burst release helps to control the rapid initial growth of bacteria.

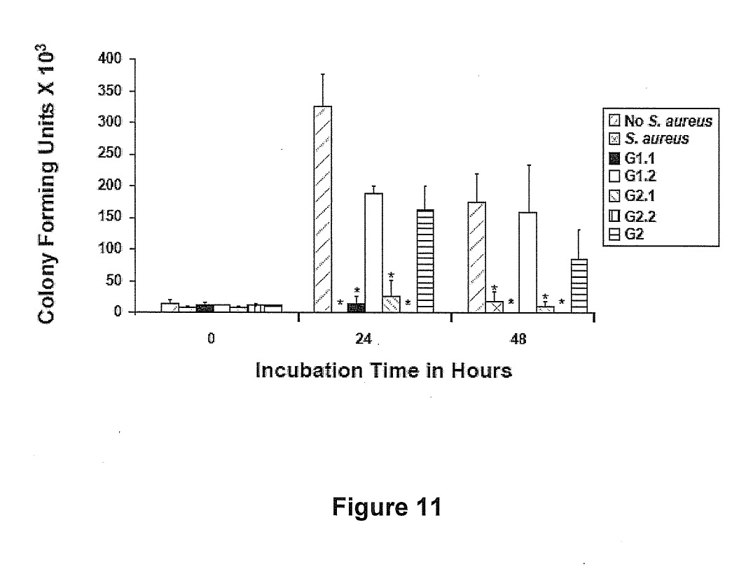

The viability of intracellular S. aureus following treatment with nanoparticles was determined. S. aureus was allowed to infect cultured primary mouse osteoblasts as described herein, followed by removal and killing of all remaining extracellular bacteria in culture. As shown in FIG. 11, following 48 hours of incubation, G1.2 and G2.2 formulations of nanoparticles loaded with nafcillin killed all intracellular bacteria. None of the nanoparticle preparations had any effect on osteoblast viability. All loaded formulations of particles caused a significant decrease in the viability of intracellular S. aureus inside infected osteoblasts at both 24 and 48 hours. Results using nafcillin alone were indistinguishable from untreated S. aureus-infected osteoblasts, confirming nafcillin alone cannot penetrate the eukaryotic cells.

FIG. 11 is a bar graph showing viability of intracellular S. aureus in osteoblasts treated with nanoparticles or nanoparticles loaded with nafcillin. Primary mouse osteoblasts were infected intracellularly with S. aureus and were subsequently treated with unloaded nanoparticles (G1 and G2) or nanoparticles loaded with nafcillin (G1.1, 1.2, 2.1 and 2.2) for 1 (0 hour), 24 and 48 hours. Osteoblasts were subsequently lysed following these time intervals with a solution containing 0.1% triton X-100, and serial dilutions of the lysates were plated on tryptic soy agar (TSA). Following an overnight incubation at 37° C., numbers of viable bacteria were enumerated, *, P<0.05 versus S. aureus-treated osteoblasts at the same time point. As noted above, following 48 hours of incubation, the G1.2 and G2.2 formulations of nanoparticles loaded with nafcillin killed all intracellular bacteria. All loaded formulations of particles caused a significant decrease in the viability of intracellular S. aureus inside infected osteoblasts at both 24 and 48 hours.

### Example 3

### Formulations for Nanoparticle Antibiotic-Delivery Systems

This Example provides nanoparticle antibiotic-delivery systems to be employed in the following Examples 4 and 5. One exemplary system degrades and releases drug rapidly (targeting extracellular bacteria) and one exemplary system degrades and releases drug slowly (targeting intracellular bacteria). Three different broad-spectrum antibiotics are used, yielding a total of six distinct nanoparticle-antibiotic conjugates.

Based on successful studies utilizing nafcillin, the presently disclosed subject matter uses the same methods to prepare slow- and fast-release nanoparticles loaded with vancomycin, tobramycin, and piperacillin-tazobactam. These drugs were chosen for the following reasons. Vancomycin has broad-spectrum activity against gram-positive bacteria including MRSA and CA-MRSA strains of S. aureus. Tobramycin has broad-spectrum activity against gram-negative bacteria. The combination of piperacillin-tazobactam has broad-spectrum activity against both gram-positive and gram-negative bacteria including anaerobes. Each of these three drugs is effective against extracellular bacteria, but none can readily penetrate into eukaryotic cells to gain access to intracellular organisms. A spray device contains all 6 nanoparticle-antibiotic conjugates.

The basic steps to be taken are the following:

1. Synthesis of different polyanhydrides for fast- and slow-release of loaded antibiotics (those noted above).

2. Preparation of nano-, submicron, and micron size particles from biodegradable and biocompatible FDA-approved polymers.

3. Characterization of the above particles.

4. In vitro and in vivo studies of release profiles of antibiotics in the extracellular and intracellular environments (see Examples 4 and 5 immediately below).

Based on the microstructure and molecular weights and size of the nanoparticles in some embodiments they are able to immediately release antibiotics in high doses especially extracellulary in soft tissue. Sustained release of antibiotics especially intracellularly is provided through nano- and sub micron sized particles which can enter eukaryotic cells and release antibiotics as demonstrated herein above.

The following materials and methods section describes the synthesis of biodegradable polymers, the preparation of varying size scale particles, their characterizations and release profiles.

Synthesis of Poly (1,3-bis(p-carboxy-phenoxy propane):sebacic acid): Poly (1,3-bis(p-carboxy-phenoxy propane):sebacic acid) (Poly (CPP:SA) is prepared from prepolymers. Poly (1,3-bis(p-carboxy-phenoxy propane) and polysebacic acid are the prepolymers. Prepolymers and co-polymers are characterized by GPC, DSC, 1HNMR, and ATR-FTIR.

Preparation of Poly (1,3-bis(p-carboxy-phenoxy propane):sebacic acid) (Poly (CPP:SA)) nanoparticles: Nanoparticles are prepared by oil-in-water emulsion solvent extraction/evaporation technique. Polyvinyl alcohol is used as surfactant. The co-polymer is dissolved in dichloromethane and PVA in deionized water. Polymer solution (1 mL) is mixed with 10 mL aqueous PVA solution under sonication. Solvent is evaporated from the emulsion and nanoparticles are collected and purified by ultracentrifugation. Lyophilization results in a dry powder. Particle size and zeta potential (surface charge) of nanoparticles is determined, and differential scanning calorimetry of nanoparticles is used to study the degree of crystallinity of nanoparticles.

Preparation of Poly (1,3-bis(p-carboxy-phenoxy propane):sebacic acid (Poly (CPP:SA)) microparticles with and without antibiotics: Poly (CPP:SA) microparticles containing antibiotics are prepared by a spray-drying method. Co-polymer is dissolved in methylene chloride and antibiotics powders are dispersed in this solution. The dispersion is sonicated for 5 minutes prior to spray drying in a Buchi Mini Dryer (available from Buchi, New Castle, Del., United States of America).

Alternative procedures are as follows: One gm poly (CPP-SA) (80:20), MW=16,000, are dissolved in 1 mL methylene chloride. Drug is suspended in the solution, mixed, dropped in silicon oil containing 1-5% of span 85 and stirred at a known stirring rate using an overhead stirrer type RZR50, “CAFRAMO” and a 3 blade impeller. After 1 hour, petroleum ether is introduced and stirring continued for another hour. The microspheres are isolated by filtration, washed with petroleum ether, dried overnight in a lyophilizer, sieved (using US standard sieve series) and stored in a freezer. A different method is applied for prepolymers with higher molecular weight. Two gm of polymer is dissolved in 10 mL of methylene chloride. Drug is added and the mixture suspended in silicon oil containing span 85 and a known amount of methylene chloride. The amount of methylene chloride depends on the type and molecular weight of the polymer used. (Poly (CPP-SA) (80:20), MW 30,000-40,000 silicon oil-methylene chloride 4:1; Poly (CPP-SA) (50:50),MW 40,000 silicon oil-methylene chloride 1:1). After dropping the polymer solution into the silicon oil petroleum ether is added and stirring continued for 2 hours. The microspheres are isolated by filtration, washed with petroleum ether, dried overnight in a lyophilizer and stored in a freezer.

Antibiotic release profiles: Antibiotic release profiles from nanoparticles and microparticles are studied by UV spectroscopic and fluorescence spectroscopic techniques.

Synthesis of PLGA nanoparticles with antibiotics: PLGA nanoparticles are prepared by oil-in-water emulsion solvent extraction/evaporation technique. Polyvinyl alcohol is used as surfactant. Polymer is dissolved in dichloromethane and mixed with an aqueous solution of antibiotics by vortexing. This dispersion is added to an aqueous solution of surfactant. Nanoparticles are collected and purified by ultracentrifugation, followed by lyophilization.

Antibiotic release profiles: Antibiotic release profiles from nanoparticles and microparticles are studied by UV spectroscopic and Fluorescence spectroscopic techniques.

Imaging of intra- and extracellular antibiotic release: This is examined via confocal microscopy as per procedures described herein. For example vancomycin-quantum dot (QD) bioconjugates are synthesized as markers for release from various particle sizes.

Synthesis of vancomycin-QD bioconjugates. The proposed scheme for the bioconjugation of QDs to vancomycin is described below. Drug release profiles are studied by UV spectroscopic and Fluorescence spectroscopic techniques.

Stealth or PEGylated nanoparticles: Block copolymers of PLGA and PAH with polyethylene glycol (PEG) blocks or surface functionalization can provide specific bioadhesive interactions. These particles thus provide dual objectives of bio-adhesion as well as unhindered transport due to “brushlayers” of the PEG. These block copolymers are synthesized and modified as per clinical requirements.

Characterization methods for polymers and particles: GPC is employed to determine the molecular weight distribution; NMR for structural information of polymer; Zeta potential for surface charge; DLS for particle size determination; and DSC for crystallinity of particles.

Vancomycin modified QD: Amine functionalized QD in MES buffer is mixed with vancomycin 1-ethyl-3-(dimethylaminopropylcarbodiimide hydrochloride; EDC) N-hydroxy succinimide for 6 hours at room temperature. The resulting vancomycin modified QD is centrifuged and then re-dispersed in MES buffer 3 times. The dispersion of Vancomycin modified QD is stored at 4° C.

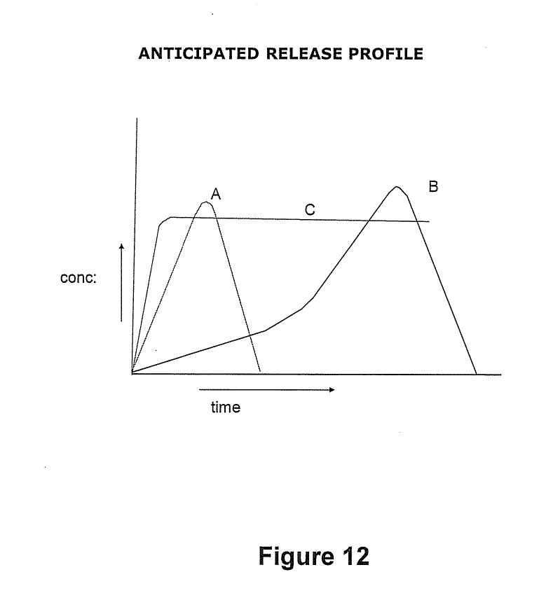

Expected Results: A series of particles is available for Example 4 and 5 presented immediately below. These ensembles contain a mixture of nano-, submicron and micron sized particles of various polymers of PLG, PLGA and PAH. A profile of such releases is shown in FIG. 12: A, a “spike” in delivery; B is slower sustained release; C is “spike” plus sustained release. Each spray ensemble is calculated based on release profiles.

### Example 4

### In Vitro Testing of Nanoparticle Antibiotic-Delivery System

This Example pertains to the testing of a nanoparticle antibiotic-delivery system of the presently disclosed subject matter in vitro against purely extracellular and purely intracellular Staphylococcus aureus. This bacterium is the most common cause of human bone disease, is typically resistant to multiple antibiotics, and causes both acute and chronic osteomyelitis due to its extracellular and intracellular presence. This Example is to determine if the particle mixture generated above is effective in killing one of the most common causes of soft-tissue infection, and the most common cause of human bone disease. This Example has specifically chosen arguably the organism posing the greatest challenge, given its resistance to multiple drugs, and its extracellular and intracellular life.

Bacterial strains and growth conditions for extracellular antimicrobial activity experiments: S. aureus strain UAMS-1 (ATCC 49230) (osteomyelitis clinical isolate) or S. aureus strain USA 300 (dominant CA-MRSA strain in the USA) is grown overnight in 5 ml of tryptic soy broth (TSB) at 37° C. with aeration (approximately 10⁹ colony forming units (cfu) per ml). Bacteria are diluted in TSB to 5×10⁵ cfu/ml and exposed to either no nanoparticle preparation, unloaded nanoparticles, or to nanoparticle-antibiotic conjugate preparations increasing over a range of concentrations. Bacteria are allowed to grow overnight at 37° C. with aeration. Lack of turbidity at a given nanoparticle concentration, coupled with no growth following plating on tryptic soy agar (TSA) plates with incubation at 37° C. overnight indicates killing of purely extracellular S. aureus. It is unlikely that tobramycin-loaded nanoparticles will be effective in killing either strain, since it is most effective against gram-negative bacteria.

Normal mouse osteoblast cell culture: Normal osteoblast cell cultures are prepared from mouse neonates according to a method previously described for chick embryos. Bone-forming cells are isolated from mouse neonate calvariae by sequential collagenase-protease digestion. The periostea is removed, the frontal bones harvested free of the suture regions, and the bones incubated for 10 minutes at 37° C. in 10 mL of digestion medium containing collagenase (375 units/mL, Type VII, Sigma Chemical Company, St. Louis, Mo., United States of America) and protease (7.5 units/ml, Sigma). The digestion medium and released cells is removed and discarded. Ten mL of fresh digestion medium is added, and the incubation continued for 20 minutes. Cells are harvested by centrifugation, and rinsed 3 times in 25 mM HEPES buffered, Hank's balanced salt solution (pH 7.4, HBSS). The digestion step is repeated twice, and the 3 cell isolates pooled in mouse osteoblast growth medium (OBGM) comprising Dulbecco's modified Eagle's medium containing 25 mM HEPES, 10% fetal bovine serum (Sigma), 2 g/L sodium bicarbonate, 75 μg/mL glycine, 100 μg/ml ascorbic acid, 40 ng/ml vitamin B12, 2 μg/ml p-aminobenzoic acid, 200 ng/ml biotin, and 100 U/mL-100 μg/mL-0.25 μg/ml penicillin-streptomycin-Fungizone (pH 7.4). Cells are seeded in 6-well cluster plates and incubated at 37° C. in a 5% CO₂ atmosphere until they reach confluence (6-7 days).

Bacterial strains and growth conditions for intracellular antimicrobial activity experiments: S. aureus strain UAMS-1 (ATCC 49230) (osteomyelitis clinical isolate) or S. aureus strain USA 300 (dominant CA-MRSA strain in the USA) are grown overnight in 5 mL of tryptic soy broth (TSB) at 37° C. with aeration. Bacteria are harvested by centrifugation for 10 minutes at 4300×g at 4° C. and washed in 5 mL of HBSS. Bacteria are then resuspended OBGM lacking antimicrobial agents.

Infection and invasion assay: Following resuspension in OBGM, bacterial cell density is determined via spectrophotometric analysis. Cells are then diluted in OBGM to obtain the desired multiplicity of infection (M.O.I.). Osteoblasts are infected with S. aureus at a multiplicity of infection of 25:1. Following a 45-minute infection period, osteoblasts are washed three (3) times with HBSS followed by incubation in media containing 25 μg/ml gentamicin to kill extracellular bacteria. Following incubation with gentamicin, only viable intracellular bacteria remain in the infected cultures. Infected cultures are then exposed to either no nanoparticle preparation, to unloaded nanoparticles, or to nanoparticle-antibiotic conjugate preparations increasing over a range of concentrations. Following various time points, osteoblasts are lysed by addition of 0.1% Triton X-100 with incubation for 5 minutes at 37° C. to release intracellular bacteria. Lysates are plated on TSA plates followed by incubation at 37° C. overnight. Lack of growth on the TSA plates indicates killing of intracellular S. aureus. Separate wells are used to examine osteoblast viability using a standard trypan blue exclusion assay. In preliminary studies, there was no significant effect on viability of the eukaryotic cells.

Expected results: It is expected that these experiments will determine the optimal concentration and time of exposure to antibiotic-loaded nanoparticles resulting in effective killing of purely extracellular and purely intracellular S. aureus. It is again unlikely that tobramycin-loaded nanoparticles will be effective in killing either strain, since it is most effective against gram-negative bacteria.

### Example 5

### In Vivo Testing of Nanoparticle Antibiotic-Delivery System

Nanoparticle antibiotic-delivery systems in accordance with the presently disclosed subject matter are tested in vivo using well established rat models of soft tissue and bone injury. For high-energy, severely contaminated fractures and resulting acute and chronic osteomyelitis, treatment includes aggressive surgical debridement of the infected/necrotic tissue, followed by defect reconstruction and varied lengths of systemic antibiotic treatment. Local application of antibiotic-impregnated nanoparticles should yield improved outcomes compared to control animals treated with debridement and systemic antibiotics. Contaminated soft tissue injury is also addressed in this Example, since soft tissue wound infection can also impact the final functional recovery of the patient and can lead to infection of underlying or adjacent osseous structures and orthopaedic fracture fixation hardware.

The present Example employs two (2) well-established models of infection (soft tissue and bone), and examines the effect of amount of contaminating bacterial dose, loaded nanoparticle concentration, and time of nanoparticle exposure on resulting numbers of viable bacteria. Controls include animals infected but untreated, as well as animals infected and treated with systemic antibiotics. It is expected to determine optimum topical dosage and time of exposure to loaded nanoparticles on effective reduction or elimination of viable bacteria. Even if topical application yields equivalent results to systemic therapy, topical application is believed to be superior approach to treatment, even if one only considers the deleterious effects of systemic therapy on the normal bacterial flora, including selection for resistant bacteria.

Soft Tissue Infection Model: The model described below is an extreme animal test model that does not mimic human wound care. In a human patient, the wound would be debrided, hardware might be placed, and the wound, if still contaminated, would not be closed. The model of this Example represents a “worse case” scenario.

Sprague-Dawley rats (Harlan-Sprague-Dawley, Indianapolis, Ind., United States of America), weighing between 450 and 550 g each, are used. The animals are housed in individual cages and given unrestricted access to food and water. The soft tissue infection model is a modification of that described previously (Fallon et al., “Use of cefazolin microspheres to treat localized methicillin-resistant Staphylococcus aureus infections in rats,” J. Surg. Res., Vol. 86, No. 1, pgs. 97-102 (2001)).

Rats are given subcutaneous administration of 0.05 mg/kg buprenorphine HCL and 5 mg/kg carprofen as analgesics 30 minutes prior to surgery. Rats are then anesthetized with the inhalant isoflurane during the entire surgical procedure, and animals kept warm on a heating pad during surgery. Skin preparation includes clipping the hair from the back followed by skin cleansing with povidone-iodine scrub, 70% alcohol, and povidone-iodine solution. A standard incision measuring 4 cm in length and 5 mm lateral and parallel to the vertebral column is carried through the skin. The incision is then continued to a depth of approximately a few millimeters, and then carried into the underlying paraspinous muscles. Sterile sand (100 mg) is introduced into each wound as an infection-potentiating foreign body and the wounds are inoculated with 100 WI of a Staphylococcus aureus suspension containing approximately 5×10⁷ colony forming units. Following infection periods ranging from 1-6 hours (as determined from in vitro studies disclosed herein), infection sites are either untreated, treated topically for different time periods with nanoparticle-antibiotic conjugates (6 different constructs—slow and fast release; vancomycin-, tobramycin-, or piperacillin-tazobactam-loaded), while a control group of animals receive systemic antibiotic therapy following infection at the 6-hour time point (vancomycin or tobramycin or piperacillin-tazobactam at concentrations of 15 mg/kg twice per day, 5 mg/kg once per day, and 80 mg/kg every 8 hours, respectively)(in a previous study using this dose of S. aureus, 5 rats out of 63 died within 3 days prior to antimicrobial therapy) (Fallon et al., “Use of cefazolin microspheres to treat localized methicillin-resistant Staphylococcus aureus infections in rats,” J. Surg. Res., Vol. 86, No. 1, pgs. 97-102 (2001)).

Topical treatment of animals includes loading a commercially-available spray bottle with the loaded nanoparticle suspensions, and spraying defined volumes 1× into the wound site. A commercially-available sprayer that delivers the most consistent volume delivered per spray is used. Standard microbiological practice including the use of protective eyeware and a surgical mask minimizes the risk of generation of an aerosol of S. aureus. This risk is indeed minimal, given the ability of S. aureus adhesins to bind to animal cells and tissues. The wounds are then closed with surgical staples, animals removed from the heating pad, and the animals observed for the next 5 weeks for clinical evidence of wound infection. Rats are given 0.05 mg/kg buprenorphine HCL as an analgesic every 8-12 hours for 48 hours following surgery. All of the surviving animals are anesthetized at 5 weeks as described herein above and then euthanatized by an intracardiac injection of a lethal dose of sodium pentobarbitol (150 mg/kg). The wounds are exposed using sterile technique and examined carefully for evidence of pus or abscess formation. A piece of tissue is excised from the medial aspect of each pocket, weighed, and transferred to a vial containing 2 ml of normal sterile saline. The tissue is homogenized for 1 minute and serial 10-fold dilutions are prepared and plated on blood agar. Following incubation at 37° C. overnight, the number of bacteria recovered are quantified and expressed as cfu/g of tissue.

Bone Infection Model: As with the animal model described above, it is not desired to try to duplicate the conditions of a high-energy open fracture, but rather to assess the topical particle treatment versus systemic therapy for the prevention of infection.

A rat model of staphylococcal osteomyelitis is used, since this model reproduces the clinical and gross pathological phases of inflammatory bone diseases such as human post-traumatic osteomyelitis (Marriott et al., “Osteoblasts express the inflammatory cytokine, interleukin-6, in a murine model of Staphylococcus aureus osteomyelitis and infected human bone tissue,” Am. J. Pathol., Vol. 164, pgs. 1399-1406 (2004).

Rats are given subcutaneous (SC) administration of 0.05 mg/kg buprenorphine HCL and 5 mg/kg carprofen as analgesics 30 minutes prior to surgery. Rats are then anesthetized with the inhalant isoflurane during the entire surgical procedure, animals kept warm on a heating pad during surgery, and the femur surgically exposed by blunt dissection techniques. A high speed drill with a round 2 mm burr is used to abrade the bone surface. Damaged bone sites are then either untreated or inoculated with 5×10⁷ colony forming units of S. aureus in agarose beads.

Agarose beads containing S. aureus are prepared as follows: 1.4% low melt agarose (Invitrogen, Carlsbad, Calif., United States of America) are cooled to 40-42° C. prior to the addition of bacteria. This mixture is added to mineral oil, vigorously stirred, and cooled rapidly on ice. The resulting agarose beads are washed and stored on ice prior to bone application (this is a thick slurry and is swabbed onto the bone). This method of application induces local infection in bone tissue but markedly reduces the risk of systemic bacterial infection and in the mouse model typically results in no loss of animals to infection.

It is unclear from the art whether this dose of S. aureus represents a LD50 dose for animals that are not treated with antimicrobial agents; however, at 4 days following infection via this model, bone tissue from femurs exposed to S. aureus demonstrated a high degree of disorganization characteristic of aberrant bone remodeling commonly associated with bacterial infections of bone tissue, and exhibited marked expression of the pro-inflammatory cytokine IL-6 (Bost et al., “Staphylococcus aureus infection of mouse or human osteoblasts induces high levels of interleukin-6 and interleukin-12 production,” J. Infec. Dis., Vol. 180, pgs. 1912-20 (1999)).

Infection periods range from 1-6 hours (times are determined from in vitro approaches disclosed herein), and infection sites are either untreated or treated topically with nanoparticle-antibiotic conjugates (6 different constructs- slow and fast-release; vancomycin-, tobramycin-, or piperacillin- tazobactam-loaded), while a control group of animals receive systemic antibiotic therapy following infection at the 6-hour time point as described above. Topical delivery of the loaded nanoparticles is also as described above.

The muscle fascias and surgical incision are closed, animals removed from the heating pad, and the disease allowed to proceed as described above for the soft tissue model and animals examined for clinical evidence of osteomyelitis. Rats are given 0.05 mg/kg buprenorphine HCL and 5 mg/kg carprofen as analgesics every 8-12 hours for 48 hours following surgery. All of the surviving animals are anesthetized at 5 weeks as described above and then euthanatized by an intracardiac injection of a lethal dose of sodium pentobarbitol (150 mg/kg). The wounds are exposed using sterile technique and examined carefully for evidence of pus or abscess formation. Bone tissue is transferred to a vial containing 2 mL of normal sterile saline. The tissue is homogenized for 1 min and serial 10-fold dilutions are prepared and plated on blood agar. Following incubation at 37° C. overnight, the number of bacteria recovered is quantified and expressed as cfu/g of tissue.

Statistical analysis: Mean bacterial loads (+/− S.E.M.) are compared following euthanasia in each animal model between approximately 6 different conditions (untreated, systemically-treated, +loaded nanoparticle-treated at 4 distinct time periods ranging from 1-6 hours using concentrations of particles determined from in vitro methods disclosed herein). It is also possible concentrations of loaded nanoparticles need to be increased over those used successfully in the in vitro approaches disclosed herein. Based on statistical considerations, 10 rats per group (total of 100 rats for each model) would be appropriate if the concentrations of loaded nanoparticles are effective at the levels determined in the in vitro approaches disclosed herein. Based on an assumed within-groups standard deviation of 1 log, one could detect a difference between the treatment groups of 3 logs using the usual significance level of 5%, and with 99% power with 10 animals. Because of the susceptibility of bacteria to antimicrobial agents at least a difference between treatment groups of 3 logs is expected and it would be more likely to be greater than an 8 log difference.

Expected Results: It is expected that these experiments will determine the optimum concentration and time of exposure to antibiotic-loaded nanoparticles resulting in effective killing of S. aureus in appropriate animal models of disease. Importantly, the nanoparticles loaded with nafcillin have remained stable at room temperature for over 6 months, with no loss in antimicrobial activity.

### Example 6

### Testing of Nanoparticle Antibiotic-Delivery System in Surgery and Other Settings

In addition to addressing the immediate needs of injured patients, the presently disclosed subject matter has application in routine surgical practice. The costs to society associated with extremity infections and osteomyelitis are enormous. The prevention of surgical infection is a primary focus of current patient safety initiatives. Over 2.528 million major open orthopaedic surgical procedures are performed each year. At present, all receive systemic peri-operative antibiotic therapy. A local nanoparticle-based antibiotic-delivery system targeting both extracellular and intracellular bacteria to coat the surgical wound layers during closure would reduce the post-operative infection rate, thus reducing deep infections. The presently disclosed subject matter could be applied to all surgical wounds, with or without implants.

Results from the in vitro and in vivo studies presented in the Examples above provide a strong rationale for the treatment of both wounded patients and patients undergoing surgical procedures. As noted above, nanoparticles loaded with nafcillin have remained stable at room temperature for over 6 months, with no loss in antimicrobial activity. Thus, spray devices could be carried without refrigeration on emergency vehicles and used by first responders.

Clinical efficacy and follow-on effectiveness studies are employed to determine the safety and clinical significance of the presently disclosed antibiotic nanoparticle delivery compositions and methods. A primary clinical trial employs a prospective randomized controlled trial comparing local and systemic infectious outcomes of patients with severe extremity injuries treated with standard systemic antibiotic therapy compared to patients whose injuries are only treated by topical local therapy. A secondary clinical trail tests the presently disclosed antibiotic nanoparticle delivery compositions and methods against systemic therapy in the treatment of acute and chronic osteomyelitis and major soft tissue infections. Another clinical trail assesses the impact of antibiotic-loaded nanoparticles as possible prophylactic agents, to be delivered directly to a high-risk surgical wound at the time of wound closure.

### REFERENCES

All references cited herein above or listed below, including patents, patent applications, and scientific literature, are incorporated herein by reference in their entireties to the extent that they supplement, explain, provide a background for, or teach methodology, techniques, and/or compositions employed herein.

Alexander et al. (2003) BMC Microbiol., Vol. 3, pg. 5.

Astete et al. (2006) J. Biomater. Sci. Polymer Edn., Vol. 17, pgs. 247-289.

Bruchez et al. (1998) Science, Vol. 281, pgs. 2013-2016 (1998).

Calhoun et al. (1997) Clin Orthopaed Related Res, Vol. 341, pgs. 206-214.

de Faria et al. (2005) Macromol. Symp., Vol. 229, pgs. 228-233.

Ellington et al. (1999) Microb. Pathog., Vol. 26, pgs. 317-323.

Ellington et al. (2003) J. Bone Joint Surg. Br., Vol. 85, pgs. 918-21.

Gonsalves et al. (1998) Biomaterials, Vol. 19, pgs. 1501-1505.

Hans et al. (2002) Solid State Mater. Sci., Vol. 6, pgs. 319-327.

Jaiswal et al. (2003) Nature Biotechnol., Vol. 21, pgs. 47-51.

Kumar et al. (2004) Biomaterials, Vol. 25, pgs. 1771-1777.

Kwon et al. (2001) Colloids Surf A, Vol. 182, pgs. 123-130.

Lagerholm et al. (2004) Nanoletters, Vol. 4, pgs. 2019-2022.

Lamprecht et al. (1999) Intl. J. Pharmaceut., Vol. 184, pgs. 97-105.

Larson et al. (2003) Science, Vol. 300, pgs. 1434-1436.

Laurencin et al. (2001) Biomaterials, Vol. 22, pgs. 1271-1277.

Lucke et al. (2003) J. Biomed. Mater. Res. B Appl. Biomater., Vol. 67, pgs. 593-602.

Mandal et al. (2002) Pharmaceut. Res., Vol. 19, pgs. 1713-1719.

Panyam et al. (2003) Adv. Drug Deliv. Rev., Vol. 55, pgs. 329-347.

Rosenthal et al. (2002) J. Am. Chem. Soc., Vol. 124, pgs. 4586-4594.

Song et al. (1997) J. Controlled Release, Vol. 43, pgs. 197-212.

Soppimath et al. (2001) J. Controlled Release, Vol. 70, pgs. 1-20.

U.S. Patent Application Publication No. 20970292524.

Varela et al. (2001) J. Chem. Physics, Vol. 114, pgs. 7682-7687.

Zweers et al. (2006) J. Controlled Release, Vol. 114, pgs. 317-324.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A composition comprising an antimicrobial agent and a biodegradable delivery vehicle, wherein the biodegradable delivery vehicle is adapted to enter a cell and release the antimicrobial agent in the cell as it biodegrades.

2. The composition of claim 1, wherein the biodegradable delivery vehicle comprises a nanoparticle.

3. The composition of claim 3, wherein the biodegradable delivery vehicle comprises poly(lactic acid), poly(lactide-co-glycolide), polyanhydride, poly(1, 3-bis-(carboxyphenoxypropane):sebacic acid, or a combination thereof.

4. The composition of claim 1, wherein the antimicrobial agent is selected from the group consisting of vancomycin and nafcillin.

5. The composition of claim 1, wherein the biodegradable delivery vehicle comprises at least two different antimicrobial agents.

6. The composition of claim 1, wherein the composition is formulated for local administration to a site of infection, optionally topical administration.

7. The composition of claim 1, further comprising a pharmaceutically acceptable carrier, diluent, or excipient.

8. An article of manufacture comprising a biodegradable delivery vehicle comprising an antimicrobial agent, packaged in a hermetically sealed, sterile container, the container having a label affixed thereto, the label bearing printed material identifying the antimicrobial agent and providing information useful to an individual administering the biodegradable delivery vehicle to a subject in need thereof, wherein the biodegradable delivery vehicle is adapted to enter infected cells to thereby kill intracellular microbes present therein.

9. The article of manufacture of claim 8, wherein the hermetically sealed, sterile container further comprises a pharmaceutically acceptable carrier, diluent, or excipient or further comprises one or more substances that when a sterile liquid is added to the hermetically sealed, sterile container reconstitutes a pharmaceutically acceptable carrier, diluent, or excipient.

10. A method for treating a microbial infection at a pre-determined site in a subject, or the possibility of a microbial infection, wherein the microbial infection is characterized by intracellular presence of a microbe in a subject, the method comprising administering to the subject at the pre-determined site a composition comprising an antimicrobial agent complexed to and/or within a biodegradable delivery vehicle, and further wherein the biodegradable delivery vehicle is adapted to enter a cell and release the antimicrobial agent in the cell as it biodegrades to thereby kill intracellular microbes if present in the cells.

11. The method of claim 10, wherein the microbial infection comprises a bacterial infection.

12. The method of claim 10, wherein the bacterial infection comprises a S. aureus infection.

13. The method of claim 24, wherein the cell is a bone cell, optionally an osteoblast.

14. A composition comprising a first delivery vehicle comprising a first antimicrobial agent and a second delivery vehicle comprising a second antimicrobial agent, wherein the first delivery vehicle is adapted to release the first antimicrobial agent at a rate that differs from that at which the second delivery vehicle releases the second antimicrobial agent.

15. The composition of claim 14, wherein the first delivery vehicle, the second delivery vehicle, or both comprises a nanoparticle.

16. The composition of claim 15, wherein the first delivery vehicle, the second delivery vehicle, or both comprises poly(lactic acid), poly(lactide-co-glycolide), polyanhydride, poly(1, 3-bis-(carboxyphenoxypropane):sebacic acid, or a combination thereof.

17. The composition of claim 14, wherein the first and the second delivery vehicles comprise parts of a single structure.

18. The composition of claim 14, wherein at least one of the first antimicrobial agent and the second antimicrobial agent is selected from the group consisting of vancomycin and nafcillin.

19. The composition of claim 14, wherein the first antimicrobial agent and the second antimicrobial agent are the same.

20. The composition of claim 14, wherein the composition is formulated for local administration to a site of infection, optionally topical administration.

21. The composition of claim 14, further comprising a pharmaceutically acceptable carrier, diluent, or excipient.

22. An article of manufacture comprising a first delivery vehicle comprising a first antimicrobial agent and a second delivery vehicle comprising a second antimicrobial agent, packaged in a hermetically sealed, sterile container, the container having a label affixed thereto, the label bearing printed material identifying the first antimicrobial agent and/or the second antimicrobial agent and providing information useful to an individual administering the first and second delivery vehicles to a subject in need thereof, wherein the first delivery vehicle is adapted to degrade rapidly in vivo in order to kill extracellular microbes, if any, present at a site on and/or in the subject, and the second delivery vehicle is adapted to enter infected cells present at the site, to thereby kill intracellular microbes.

23. The article of manufacture of claim 22, wherein the hermetically sealed, sterile container further comprises a pharmaceutically acceptable carrier, diluent, or excipient or further comprises one or more substances that when a sterile liquid is added to the hermetically sealed, sterile container reconstitutes a pharmaceutically acceptable carrier, diluent, or excipient.

24. A method for treating a pre-determined site in a subject for a microbial infection, or the possibility of a microbial infection, wherein the microbial infection is characterized by both extracellular and intracellular presence of a microbe in a subject, the method comprising administering to the subject at the pre-determined site a composition comprising:
(a) a first delivery vehicle comprising a first antimicrobial agent; and
(b) a second delivery vehicle comprising a second antimicrobial agent,
wherein the first delivery vehicle is adapted to deliver the first antimicrobial agent in a burst in order to kill extracellular microbes, if present at the site, and the second delivery vehicle is adapted to enter cells present at the site, to thereby kill intracellular microbes if present in the cells.

25. The method of claim 24, wherein the microbial infection comprises a bacterial infection.

26. The method of claim 25, wherein the bacterial infection comprises a S. aureus infection.

27. The method of claim 24, wherein the site of infection comprises a bone cell, optionally an osteoblast.

28. A method for treating a pre-determined site in a subject for a microbial infection, or the possibility of a microbial infection, the method comprising administering to the subject at the predetermined site a composition comprising an antimicrobial agent complexed to and/or within a nanoparticle.

29. The method of claim 28, wherein the microbial infection comprises a bacterial infection.

30. The method of claim 29, wherein the bacterial infection comprises a S. aureus infection.

31. The method of claim 28, wherein the site of infection comprises a bone cell, optionally an osteoblast.

32. The method of claim 28, wherein the composition is formulated for local administration to a site of infection, optionally topical administration.

33. The method of claim 28, wherein the nanoparticle comprises poly(lactic acid), poly(lactide-co-glycolide), polyanhydride, poly(1, 3-bis-(carboxyphenoxypropane):sebacic acid, or a combination thereof.

34. The method of claim 28, wherein the antimicrobial agent is selected from the group consisting of vancomycin and nafcillin.

35. The method of claim 28, wherein the biodegradable delivery vehicle comprises at least two different antimicrobial agents.

36. An implantable structure comprising a composition according to claim 1.

37. An implantable structure comprising a composition according to claim 14.
